(19) United States
(12) Patent Application Publication
(10) Pub. No.: US 2020/0017913 A1
(43) Pub. Date: Jan. 16, 2020

(54) METHODS AND SYSTEMS FOR PREDICTING TREATMENT RESPONSES IN SUBJECTS

(71) Applicant: PRESIDENT AND FELLOWS OF HARVARD COLLEGE, Cambridge, MA (US)

(72) Inventors: Andrew L. BEAM, Boston, MA (US); Isaac S. KOHANE, Newton, MA (US); Nathan PALMER, Somerville, MA (US)

(73) Assignee: PRESIDENT AND FELLOWS OF HARVARD COLLEGE, Boston, MA (US)

(21) Appl. No.: 16/489,634

(22) PCT Filed: Mar. 2, 2018
(86) PCT No.: PCT/US2018/020586

§ 371 Date: Aug. 28, 2019

Related U.S. Application Data
(60) Provisional application No. 62/466,213, filed on Mar. 2, 2017.

(51) Int. Cl. C12Q 1/6883; G01N 33/50; G16B 20/00; G16B 25/20; G16H 50/30; G06F 17/15; G06F 17/18; G16B 40/10

(52) U.S. Cl. CPC C12Q 1/6883; G01N 33/5005; G16B 20/00; G16B 25/20; C12Q 2600/106; G06F 17/153; G06F 17/18; G16B 40/10; G16H 50/30

(57) ABSTRACT

Embodiments of various aspects described herein are directed to methods, systems, and computer readable media for predicting response of cells in vitro or in vivo (e.g., in a subject) to at least one or more agents (e.g., a library of agents). Methods, systems, and computer readable media described herein generally involve a computational algorithm to predict an expected post-treatment genome-wide expression profile of a cell or subject induced by an agent. The expected post-treatment genome-wide expression can be computed as a function of a pre-treatment genome-wide expression profile of the subject and known effects of the agent on gene expression in cells. Methods, systems, and computer readable media described herein can be used for drug repositioning, to select an appropriate treatment for a diseased subject, and/or to identify responsive subjects for a particular treatment.

### CROSS REFERENCE TO RELATED APPLICATION

This application claims priority to U.S. Provisional Application No. 62/466,213, filed Mar. 2, 2017, titled “Methods and Systems for Predicting Treatment Responses in Subjects,” the contents of which are incorporated here by reference.

### TECHNICAL FIELD

Described herein relates generally to methods, systems, and computer readable media for predicting response of cells to at least one or more agents. In some embodiments, the methods, systems, and computer readable media described herein can be used to select an appropriate treatment for a diseased subject or to identify responsive subjects for a particular treatment. In some embodiments, the methods, systems, and computer readable media described herein can be used for drug repositioning.

### BACKGROUND

Drug development is expensive and slow. Cell-based assays are widely used to determine effects of active agents on gene expression levels in cells. However, only 10% of new drugs will typically move on and eventually receive regulatory approval. Hay et al. “Clinical development success rates for investigational drugs” Nat Biotechnol., (2014) 32(1): 40-51. Accordingly, there is a need for a more efficient method for drug development and/or discovery that can quickly and accurately assess the therapeutic spectrum of a large number of drugs.

### SUMMARY

Embodiments of various aspects described herein are directed to methods, systems, and computer readable media for predicting or determining response of cells in vitro or in vivo (e.g., in a subject) to at least one or more agents (e.g., a library of agents). Unlike the existing methods where cell-based assays are performed to predict or determine effects of a test agent on the cells, the methods, systems, and computer readable media described herein generally involve a computational algorithm to determine, for each test agent, an expected post-treatment gene expression profile of a cell or a subject induced by the test agent. The expected post-treatment gene expression profile can be computed as a function of a pre-treatment gene expression profile of the cell, or the subject and known effects of the test agent on gene expression in cells. Instead of performing differential gene expression data analysis as in traditional cell-based assays, the methods, systems, and computer readable media described herein account for interactions between genes in a more global, genome-wide perspective. Rather than predicting the suitable drug, this method ranks all drugs with respect to their ability to return an individual to a healthy state. This allows tradeoffs between efficacy and side effects addressed explicitly by the treating physician.

In one aspect, the inventors have developed a computational algorithm for predicting individualized response to a test agent at the gene expression level. The computational algorithm was validated by applying it to patients with inflammatory bowel disease (IBD) using drugs that are routinely used in treatment of IBD (e.g., azathioprine and sulfasalazine) to show that the computed expected post-treatment gene expression profile moves closer to a normal gene expression profile. The computational algorithm was also validated in a separate, publicly available dataset, in which gene expression profiles of colon biopsies of IBD patients before and after treatment were available. The expected post-treatment gene expression profiles (GEPs) computed using the computational algorithm agreed well with the GEPs of colon biopsies of the corresponding IBD patients after treatment, indicating that the computational algorithm can robustly predict drug response. The inventors have also shown that the computational algorithm can be used to predict a subset of IBD patients would be more likely to benefit from prednisone, while other would not. Accordingly, the methods, systems, and computer readable media described herein can be used to select an appropriate treatment for a diseased subject or identify responsive subjects for a particular treatment. In some embodiments, the methods, systems, and computer readable media described herein can be used for drug repositioning.

In one aspect, a method of selecting a treatment for a subject with a disease or disorder is described herein. The method comprises:

(i) assaying a sample from a subject with a disease or disorder to determine a pre-treatment genome-wide expression profile of the subject;
(ii) in a specifically-programmed computer, computing, for each of a library of gene-expression-modifying agents, an expected post-treatment genome-wide expression profile of the subject as a function of the pre-treatment genome-wide expression profile of the subject and known effects of the corresponding gene expression-modifying agent on gene expression in cells; and
(iii) identifying a gene expression-modifying agent as an agent that is more likely to produce a therapeutic effect on the subject when deviation of the expected post-treatment genome-wide expression profile from a normal genome-wide expression profile is smaller than deviation of the pre-treatment genome-wide expression profile from the normal genome-wide expression profile; or identifying a gene expression-modifying agent as an agent that is less likely to produce a therapeutic effect on the subject when deviation of the expected post-treatment genome-wide expression profile from a normal genome-wide expression profile is greater than or substantially same as deviation of the pre-treatment genome-wide expression profile from the normal genome-wide expression profile, thereby selecting a treatment comprising a gene expression-modifying agent that is personalized to the subject.

In some embodiments, the method can further comprise administering to the subject a gene expression-modifying agent identified to be more likely to produce a therapeutic effect on the subject. In some embodiments, the administered gene expression-modifying agent is not clinically known to be indicated for treatment of the disease or disorder that the subject is diagnosed with.

In some embodiments, the method can further comprise administering to the subject an alternative treatment when the gene expression-modifying agent is identified to be less likely to produce a therapeutic effect on the subject.

A method of treating a subject with a disease or disorder is also described herein. The method comprises: administering to a subject with a disease or disorder a treatment that is computationally selected to be more likely to modulate the genome-wide expression profile of the subject toward a normal genome-wide expression profile, wherein the computational drug selection process comprises:

(i) in a specifically-programmed computer, computing, for each of a library of gene-expression-modifying agents, an expected post-treatment genome-wide expression profile of the subject as a function of a pre-treatment genome-wide expression profile of the subject and known effects of the corresponding gene expression-modifying agent on gene expression in cells; and
(ii) identifying a gene expression-modifying agent as an agent that is more likely to produce a therapeutic effect on the subject when deviation of the expected post-treatment genome-wide expression profile from a normal genome-wide expression profile is smaller than deviation of the pre-treatment genome-wide expression profile from the normal genome-wide expression profile; or identifying a gene expression-modifying agent as an agent that is less likely to produce a therapeutic effect on the subject when deviation of the expected post-treatment genome-wide expression profile from a normal genome-wide expression profile is greater than or substantially same as deviation of the pre-treatment genome-wide expression profile from the normal genome-wide expression profile.

In some embodiments, the treatment can comprise at least one gene expression-modifying agent that is identified in the computational drug selection process to be more likely to produce a therapeutic effect.

In some embodiments, the treatment can comprise a combination treatment. The combination treatment can comprise at least two gene expression-modifying agents, wherein the combination treatment is identified in the computational drug selection process to be more likely to produce a therapeutic effect.

In some embodiments, the gene expression-modifying agent included in the treatment is not clinically known to be indicated for treatment of the disease or disorder that the subject is diagnosed with.

Another aspect relates to a method of identifying a subject who is diagnosed with a disease or disorder and is more likely to respond to a treatment. The method comprises:

(i) assaying a sample from the subject to determine a genome-wide expression profile of the subject;
(ii) in a specifically-programmed computer, computing an expected post-treatment genome-wide expression profile of the subject as a function of the pre-treatment genome-wide expression profile of the subject and known effects of the treatment on gene expression in cells; and
(iii) identifying the subject to be more likely to respond to the treatment when deviation of the expected post-treatment genome-wide expression profile from a normal genome-wide expression profile is smaller than deviation of the pre-treatment genome-wide expression profile from the normal genome-wide expression profile; or
identifying the subject to be likely to respond to an alternative treatment when deviation of the expected post-treatment genome-wide expression profile from a normal genome-wide expression profile is greater than or substantially same as deviation of the pre-treatment genome-wide expression profile from the normal genome-wide expression profile.

A further aspect relates to a method of drug repositioning. The method comprises:

(i) obtaining individual genome-wide expression profiles of patients identified with the same disease or disorder;
(ii) in a specifically-programmed computer, for each identified patient, computing an expected post-treatment genome-wide expression profile of the identified patient as a function of the corresponding individual genome-wide expression profile and known effects of a therapeutic agent on gene expression, wherein the therapeutic agent is not clinically known to be indicated for treatment of the disease or disorder identified in the patients; and
(iii) identifying the therapeutic agent as an agent that is likely to produce a therapeutic effect on the disease or disorder identified in the patients when at least 50% or more of the patients show the expected post-treatment genome-wide expression profile with a smaller deviation from a normal genome-wide expression profile than that of the individual genome-wide expression profile from the normal genome-wide expression profile, thereby computationally repositioning the therapeutic agent for a new indication; or
identifying the therapeutic as an agent that is not likely to produce a therapeutic effect on the disease or disorder identified in the patients when less than 50% of the patients show the expected post-treatment genome-wide expression profile with a smaller deviation from a normal genome-wide expression profile than that of the individual genome-wide expression profile from the normal genome-wide expression profile.

In some embodiments, the method can further comprise, when the therapeutic agent is computationally repositioned for a new indication, contacting cells in vitro or in an animal model with the therapeutic agent to experimentally validate its therapeutic effect. The cells in vitro or in animal model correspond to a model of the same disease or disorder as identified in the patients.

Another aspect relates to a method of identifying a potential adverse effect of a treatment in a subject with a disease or disorder. The method comprises:

(i) assaying a sample from the subject to determine a pre-treatment genome-wide expression profile of the subject;
(ii) in a specifically-programmed computer, computing an expected post-treatment genome-wide expression profile of the subject as a function of the pre-treatment genome-wide expression profile of the subject and known effects of the treatment on gene expression in cells; and
(iii) identifying the treatment to be more likely to induce an adverse effect in the subject when deviation of the expected post-treatment genome-wide expression profile from a normal genome-wide expression profile is larger than deviation of the pre-treatment genome-wide expression profile from the normal genome-wide expression profile, and/or the expected post-treatment genome-wide expression profile of the subject is similar to expected post-treatment genome-wide expression profiles of patients who have suffered from at least one adverse effect upon administration of the same treatment; or
identifying the treatment to be less likely to induce an adverse effect when deviation of the expected post-treatment genome-wide expression profile from a normal genome-wide expression profile is smaller than deviation of the pre-treatment genome-wide expression profile from the normal genome-wide expression profile, and/or the expected post-treatment genome-wide expression profile of the subject is different from expected post-treatment genome-wide expression profiles of patients who have suffered from at least one adverse effect upon administration of the same treatment.

In some embodiments of various aspects described herein, the computation involved in the methods can comprise principal component analysis (PCA) of the pre-treatment genome-wide expression profile and the normal genome-wide expression profile to identify a set of gene signatures that are associated with the disease or disorder identified in the subject.

In some embodiments of various aspects described herein, the gene expression-modifying agents can be selected for the computing based on their known properties to modulate expression of at least one of the gene signatures toward its corresponding expression level in normal cells not affected by the disease or disorder.

While the expected post-treatment genome-wide expression profile of the subject can be computed as a linear or non-linear function, in some embodiments, the expected post-treatment genome-wide expression profile of the subject can be computed using the following equation:

g_{new}=g+g·dⱼ

wherein g is a gene expression vector reflecting at least a subset of genes of the pre-treatment genome-wide expression profile of the subject, wherein the subset of genes correspond to the gene signatures associated with the disease or disorder; dⱼ is a transformation matrix reflecting a known modulation of gene expression in cells associated with each of the gene expression-modifying agents; and g_{new} is a gene expression matrix reflecting an expected post-treatment genome-wide expression profile for each of the gene expression-modifying agents.

The sample used to determine a genome-wide expression profile of the subject(s) in the methods described herein can be assayed by any methods known in the art. For example, the sample can be assayed by a method comprising polymerase chain reaction (PCR), a real-time quantitative PCR, microarray, RNA sequencing, and/or nucleic acid sequencing.

The methods of various aspects described herein can be applied to any disease or disorder. For example, in one embodiment, the methods described herein can be applied to an inflammatory bowel disease.

### BRIEF DESCRIPTION OF THE DRAWINGS

FIG. 1 is PCA projection of pre-treatment gene expression profile (GEP) for individuals with irritable bowel disease (IBD) such as ulcerative colitis (UC) and Crohn's disease (CD), as compared to healthy controls. Healthy patients are shown in as circles, individuals with Crohn's Disease (CD) are shown with crosses, and those with Ulcerative Colitis (UC) are shown with squares.

FIG. 2 is a schematic diagram showing an in silico method of predicting response of a diseased individual to one or more selected agent(s) at gene expression levels.

FIG. 3 is a PCA projection of predicted changes in GEP induced by azathioprine in IBD patients such as ulcerative colitis (UC) and Crohn's disease (CD). Azathioprine is a drug commonly used for treatment of IBD. The predicted trajectory of IBD patients (squares, crosses) moves towards the healthy patients (circles).

FIG. 4 is a PCA projection of predicted changes in GEP induced by sulfasalzine in IBD patients. Sulfasalzine is a drug commonly used for treatment of IBD. The predicted trajectory of IBD patients (red) moves towards the healthy patients (black).

FIG. 5 is a PCA projection of predicted changes in GEP induced by prednisone in IBD patients. Prednisone is a drug that can be used for treatment of IBD. The predicted trajectory of some IBD patients (red) moves towards the healthy patients (black), while most of the IBD patients (red) appear to move away from the healthy patients (black). This indicates that prednisone is only effective in a sub-population of IBD patients with certain gene expression profiles. Thus, the methods and systems described herein can be used to select an appropriate treatment for a subject, or to identify an effective treatment for a sub-population of patients with a disease or disorder, or to identify a subset of patients with a disease or disorder who will be more likely to respond to a specific treatment.

FIG. 6 is a schematic diagram showing an exemplary system for use in the methods described herein, e.g., for predicting response of at least one subject to one or more agents at a gene expression level.

FIG. 7 is a block diagram showing an exemplary system for use in the methods described herein, e.g., for predicting response of at least one subject to one or more agents at a gene expression level.

FIG. 8 is an exemplary set of instructions on a computer readable storage medium for use with the systems described herein.

FIG. 9 shows a table generated according to an embodiment of the present disclosure where various drugs are scored to compare an optimal vector of movement towards healthy patient data and a drug vector of movement after a patient takes the drug.

FIG. 10 is an exemplary PCA according to an embodiment of the present disclosure of predicted changes in GEP induced by trimethobenzamide in Patient #1. PCAs can be made for all the other drugs listed in FIG. 9 as well. Referring back to FIG. 10, the predicted trajectory of Patient #1, shown by the squares, moves towards the healthy patients (circles). Other sick patients are contained in a reference set and analyzed to see their predicted trajectories as well.

### DETAILED DESCRIPTION OF THE INVENTION

Embodiments of various aspects described herein are directed to methods, systems, and computer readable media for predicting or determining response of cells in vitro or in vivo (e.g., in a subject) to at least one or more agents (e.g., a library of agents). Unlike the existing methods where cell-based assays are performed to predict or determine effects of a test agent on the cells, the methods, systems, and computer readable media described herein generally involve a computational algorithm to determine, for each test agent, an expected post-treatment gene expression profile of a cell or a subject induced by the test agent. The expected post-treatment gene expression profile can be computed as a function of a pre-treatment gene expression profile of the cell or the subject and known effects of the test agent on gene expression in cells. Instead of performing differential gene expression data analysis as in traditional cell-based assays, the methods, systems, and computer readable media described herein account for interactions between genes in a more global, genome-wide perspective.

In one aspect, the inventors have developed a computational algorithm for predicting individualized response to a test agent at the gene expression level. The computational algorithm was validated by applying it to patients with inflammatory bowel disease (IBD) using drugs that are routinely used in treatment of IBD (e.g., azathioprine and sulfasalazine) to show that the computed expected post-treatment gene expression profile moves closer to a normal gene expression profile. The computational algorithm was also validated in a separate, publicly available dataset, in which gene expression profiles of colon biopsies of IBD patients before and after treatment were available. The expected post-treatment gene expression profiles (GEPs) computed using the computational algorithm agreed well with the GEPs of colon biopsies of the corresponding IBD patients after treatment, indicating that the computational algorithm can robustly predict drug response. The inventors have also shown that the computational algorithm can be used to predict a subset of IBD patients would be more likely to benefit from prednisone, while other would not. Accordingly, the methods, systems, and computer readable media of various aspects described herein can be used to select an appropriate treatment for a diseased subject or identify responsive subjects for a particular treatment. In some embodiments, the methods, systems, and computer readable media described herein can be used for drug repositioning.

The methods, systems, and computer readable media of various aspects described herein can be applied to any disease or disorder. For example, in one embodiment, the methods, systems, and computer readable media described herein can be applied to an inflammatory bowel disease.

### Methods of Determining Individualized Drug Response at Gene Expression Level and Applications Thereof

Healthy people are typically characterized by a portion or subset of “gene expression space” that is distinct from their non-healthy counterparts. A disease or disorder can be characterized by a deviation (e.g., by at least about 30% or more, including, e.g., at least about 40%, at least about 50%, at least about 60%, at least about 70%, at least about 80%, at least about 90%, or more) in gene expression profile from a normal biological state. Thus, if one could identify an agent or treatment that is determined to “nudge” a patient with a condition (e.g., a disease or disorder) towards the region of gene-expression space occupied by healthy patients, afflicted individuals are likely to experience some therapeutic benefit as a result. The methods, systems, and computer readable media described herein can be used to computationally estimate the direction and magnitude of this agent-induced “nudge” in an individual. One of the major principles underlying the methods, systems, and/or computer readable media described herein is that a subject's response to one or more agents or treatments is best considered from a global, genome-wide perspective, instead of the traditional dichotomous approaches, where one or several genes are individually compared to a corresponding reference level without considering the interactions between the genes. Accordingly, the methods, systems, and computer readable media described herein can be used to computationally identify an agent or treatment to reduce the deviation in gene expression profile induced by a disease or disorder from a normal biological state, or to restore the normal biological state.

In one aspect, a method or a computer implemented method of selecting a treatment for a subject with a disease or disorder is described herein. The method comprises:

(i) assaying a sample from a subject with a disease or disorder to determine a pre-treatment genome-wide expression profile of the subject;
(ii) in a specifically-programmed computer, computing, for each of a library of gene-expression-modifying agents, an expected post-treatment genome-wide expression profile of the subject as a function of the pre-treatment genome-wide expression profile of the subject and known effects of the corresponding gene expression-modifying agent on gene expression in cells; and
(iii) identifying a gene expression-modifying agent as an agent that is more likely to produce a therapeutic effect on the subject when deviation of the post-treatment genome-wide expression profile from a normal genome-wide expression profile is smaller than deviation of the pre-treatment genome-wide expression profile from the normal genome-wide expression profile; or
identifying a gene expression-modifying agent as an agent that is less likely to produce a therapeutic effect on the subject when deviation of the post-treatment genome-wide expression profile from a normal genome-wide expression profile is greater than or substantially same as deviation of the pre-treatment genome-wide expression profile from the normal genome-wide expression profile, thereby selecting a treatment comprising a gene expression-modifying agent that is personalized to the subject.

As used herein, the term “specifically-programmed computer” refers to a computer system comprising one or more processors; and memory to store one or more programs, which comprise instructions for performing one or more functions described herein. These programs or sets of instructions need not be implemented as separate software programs, procedures or modules, and thus various subsets of these modules may be combined or otherwise re-arranged in various embodiments. In some embodiments, memory may store a subset of the modules and data structures described herein. Further, memory may store additional modules and data structures not described herein.

As used herein, the term “assaying” or “assay” refers to performing a procedure where a biological and/or chemical property of a sample is measured. In some embodiments, the term “assaying” or “assay” refers to a biological assay and is a type of in vitro experiment. Assays are typically conducted to measure at least one or a plurality of (e.g., at least two or more) target biological molecules in a biological sample. Assays can be qualitative or quantitative.

The sample used to determine a genome-wide expression profile of the subject(s) in the methods described herein can be assayed by any methods known in the art. For example, the sample can be assayed by a method comprising polymerase chain reaction (PCR), a real-time quantitative PCR, microarray, RNA sequencing, and/or nucleic acid sequencing. Assays for gene expression profiling are commercially available.

The term “expression profile,” which is used interchangeably herein with “gene expression profile” refers to a dataset representing mRNA or transcript levels of a plurality of genes in a cell. An expression profile can comprise a dataset representing mRNA or transcript levels of, for example, at least about 10 genes, or at least about 50, 100, 200, 300, 400, 500, 600, 700, or more genes. Expression profiles can also comprise an mRNA level of a gene which is expressed at similar levels in multiple cells and conditions (e.g., a housekeeping gene such as GAPDH). For example, an expression profile of a subject refers to a dataset representing mRNA levels of 10, or at least about 50, 100, 200, 300, 400, 500, 600, 700, or more genes in a cell or tissue derived from the subject.

As used herein, the term “transcript” refers to an RNA molecule that is derived through the process of transcription from a DNA or a cDNA template. Transcripts can also be represented by proteins translated from RNA transcripts or cDNA molecules that are reverse-transcribed from RNA transcripts.

The term “genome-wide expression profile” refers to a dataset representing mRNA or transcript levels of a subset of a genome or transcriptome of a cell. For example, in some embodiments, a genome-wide expression profile can be a dataset representing mRNA or transcript levels of at least about 30% or more, including, e.g., at least about 40%, at least about 50%, at least about 60%, at least about 70%, at least about 80%, at least about 90%, at least about 93%, at least about 95%, at least about 98%, at least about 99%, or more, including 100%, of a genome or transcriptome of a cell.

As used herein, the term “genome” refers to all nucleic acid sequences (coding and non-coding) and elements present in a single cell. The term genome also applies to any naturally occurring or induced variation of these sequences that may be present in a mutant or disease variant of a cell. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism. For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a diseased cell can contain variable numbers of each chromosome in addition to deletions, rearrangements and amplification of any subchromosomal region or DNA sequence.

As used herein, the term “transcriptome” means a collection of RNA transcripts transcribed in a specific cell or tissue, whether coding or non-coding, and preferably contains all or substantially all of the RNA transcripts generated in the cell or tissue. These transcripts include messenger RNAs (mRNA), alternatively spliced mRNAs, ribosomal RNA (rRNA), transfer RNAs (tRNAs) in addition to a large range of other transcripts, which are not translated into protein such as small nuclear RNAs (snRNAs), antisense molecules such as short interfering RNA (siRNA) and microRNA and other RNA transcripts of unknown function. The transcriptome can also include proteins translated from the RNA transcripts within the transcriptome, which is an extension and reflection of gene transcription within the transcriptome.

As used herein, the term “pre-treatment genome-wide expression profile” refers to a dataset representing mRNA or transcript levels of a subset of a genome or transcriptome of a cell prior to exposure to a treatment. For example, in some embodiments, a pre-treatment genome-wide expression profile can be a dataset representing mRNA or transcript levels of at least about 30% or more, including, e.g., at least about 40%, at least about 50%, at least about 60%, at least about 70%, at least about 80%, at least about 90%, at least about 93%, at least about 95%, at least about 98%, at least about 99%, or more, including 100%, of a genome or transcriptome of a cell prior to exposure to a treatment.

As used herein, the term “treatment” refers to a composition comprising at least one or more (e.g., at least two, at least three, or more) gene expression-modifying agents. The term “gene expression-modifying agent” as used herein refer to an agent that can increase or decrease an mRNA or transcript level of at least one or more (including, e.g., at least 2, at least 5, at least 10 genes, or at least about 50, 100, 200, 300, 400, 500, 600, 700, or more) genes in a cell, as compared to an mRNA or transcript level in the absence of the gene expression-modifying agent. An agent includes, but is not limited to, proteins, peptides, nucleic acids (e.g., RNA, DNA, siRNA, shRNA), aptamers, small molecules, therapeutic agents, nutraceuticals, environmental stimuli (e.g., pressure, hypoxia, humidity, light, temperature (e.g., extremes in high and low temperatures), radiation), drugs, FDA-approved drugs, and any combinations thereof. In some embodiments of various aspects described herein, the gene expression-modifying agents can be selected based on their known properties to modulate expression at least one of the gene signatures toward its corresponding expression level in normal cells.

As used herein, the term “a library” generally refers to a collection, e.g., at least 10, at least 20, at least 50, at least 100, at least 200, at least 300, at least 400, at least 500, or more. In some embodiments, the term “library” can refer to at least 500 or more, including, e.g., at least 1000, at least 2000, or more. Accordingly, the term “a library of gene expression-modifying agents” refers to a collection comprising at least 10 or more, including, e.g., at least 20, at least 50, at least 100, at least 200, at least 300, at least 400, at least 500, or more, gene expression-modifying agents. In some embodiments, the term “a library of gene expression-modifying agents” refers to a collection comprising at least 500 or more, including, e.g., at least 1000, at least 2000 or more, gene expression-modifying agents.

As used herein, the term “post-treatment genome-wide expression profile” refers to a dataset representing mRNA or transcript levels of a subset of a genome or transcriptome of a cell after exposure to a treatment. For example, in some embodiments, a post-treatment genome-wide expression profile can be a dataset representing mRNA or transcript levels of at least about 30% or more, including, e.g., at least about 40%, at least about 50%, at least about 60%, at least about 70%, at least about 80%, at least about 90%, at least about 93%, at least about 95%, at least about 98%, at least about 99%, or more, including 100%, of a genome or transcriptome of a cell after exposure to a treatment.

The term “expected” as used herein refers to a post genome-wide expression profile predicted by a computational algorithm.

A gene expression-modifying agent is identified as an agent that is more likely to produce a therapeutic effect on a subject with a disease or disorder, when deviation of the post-treatment genome-wide expression profile from a normal genome-wide expression profile is smaller than deviation of the pre-treatment genome-wide expression profile from the normal genome-wide expression profile, for example, by at least 10% or more, including, e.g., at least 20%, at least 30%, at least 40%, at least 50%, at least 60%, at least 70%, at least 80%, at least 90%, or more.

As used herein, the term “deviation” refers to the difference between two genome-wide expression profiles. The difference can encompass both magnitude and direction. By way of example only, where the genome-wide expression profiles are expressed as loci on a 2-dimensional PCA plot, which is described in detail below, the deviation can be measured by the distance between the two corresponding loci.

In some embodiments where the gene expression-modifying agent is identified to be more likely to produce a therapeutic effect on the subject, the method can further comprise administering to the subject the particular gene expression-modifying agent. In some embodiments, the administered gene expression-modifying agent can be not clinically known to be indicated for treatment of the disease or disorder that the subject is diagnosed with.

As used herein, the phrase “more likely to produce a therapeutic effect” generally refers to likelihood of an agent to produce a therapeutic effect.

As used herein, the term “therapeutic effect” refers to reversal, alleviation, amelioration, inhibition, slowing down or stopping the progression or severity of a condition associated with, a disease or disorder. The term “therapeutic effect” includes reducing or alleviating at least one adverse effect or symptom of a condition, disease or disorder. “Therapeutic effect” can be characterized by reduction of one or more symptoms or clinical markers associated with a condition, disease, or disorder. Alternatively, there is a therapeutic effect if the progression of a disease is reduced or halted. That is, “therapeutic effect” includes not just the improvement of symptoms or markers, but also a cessation of at least slowing of progress or worsening of symptoms that would be expected in absence of treatment. Beneficial or desired clinical results include, but are not limited to alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “therapeutic effect” of a disease or disorder also includes providing relief from the symptoms or side-effects of the disease or disorder. The terms “therapeutic effect,” “treating,” and “treat” are used interchangeably herein with respect to a disease or disorder.

A gene expression-modifying agent is identified as an agent that is less likely to produce a therapeutic effect on the subject when deviation of the post-treatment genome-wide expression profile from a normal genome-wide expression profile is greater than or substantially same as deviation of the pre-treatment genome-wide expression profile from the normal genome-wide expression profile, for example, by at least 10% or more, including, e.g., at least 20%, at least 30%, at least 40%, at least 50%, at least 60%, at least 70%, at least 80%, at least 90%, or more. In some embodiments, a gene expression-modifying agent is identified as an agent that is less likely to produce a therapeutic effect on the subject when deviation of the post-treatment genome-wide expression profile from a normal genome-wide expression profile is greater than or substantially same as deviation of the pre-treatment genome-wide expression profile from the normal genome-wide expression profile, for example, by at least 1.1-fold or more, including, e.g., at least 2-fold, at least 3-fold, at least 4-fold, at least 5-fold, at least 6-fold, at least 7-fold, at least 8-fold, at least 9-fold, at least 10-fold, at least 100-fold, or more.

In some embodiments where the gene expression-modifying agent is identified to be less likely to produce a therapeutic effect on the subject, the method can further comprise administering to the subject an alternative treatment, e.g., a treatment without the particular gene expression-modifying agent.

A method of treating a subject with a disease or disorder is also described herein. The method comprises:

(i) administering to a subject with a disease or disorder a treatment that is computationally selected to be more likely to modulate the genome-wide expression profile of the subject toward a normal genome-wide expression profile, wherein the computational drug selection process comprises:

in a specifically-programmed computer, computing, for each of a library of gene-expression-modifying agents, an expected post-treatment genome-wide expression profile of the subject as a function of a pre-treatment genome-wide expression profile of the subject and known effects of the corresponding gene expression-modifying agent on gene expression in cells; and; and

identifying a gene expression-modifying agent as an agent that is more likely to produce a therapeutic effect on the subject when deviation of the post-treatment genome-wide expression profile from a normal genome-wide expression profile is smaller than deviation of the pre-treatment genome-wide expression profile from the normal genome-wide expression profile; or

identifying a gene expression-modifying agent as an agent that is less likely to produce a therapeutic effect on the subject when deviation of the post-treatment genome-wide expression profile from a normal genome-wide expression profile is greater than or substantially same as deviation of the pre-treatment genome-wide expression profile from the normal genome-wide expression profile.

As used herein, the term “administering” or “administer” refers to the placement of a composition into a subject by a method or route which results in at least partial localization of the composition at a desired site such that desired effect is produced. Routes of administration suitable for the methods described herein can include both local and systemic administration. Generally, local administration results in a higher amount of a therapeutic agent being delivered to a specific location (e.g., a target site to be treated) as compared to the entire body of the subject, whereas, systemic administration results in delivery of a therapeutic agent to essentially the entire body of the subject.

As used herein, the term “computationally” or “computation” or “computing” refers to mathematical calculation that requires use of a specially-programmed computer due to the sheer volume of data and/or complexity of the intended calculation. Thus, the computation cannot be done manually or in a human brain.

As used herein, the term “modulate,” when referring to modulating a genome-wide expression profile of a subject, refers to changing mRNA or transcript level of at least one or more (e.g., at least two, at least three, or more) genes present in the expression profile. In some embodiments, the change in the mRNA or transcript level of at least one or more (e.g., at least two, at least three, or more) genes can be reflected by a change in the locus representing the genome-wide expression profile on a 2-dimensional PCA plot.

In some embodiments, the treatment to be administered to a subject with a disease or disorder can comprise at least one gene expression-modifying agent that is identified in the computational drug selection process to be more likely to produce a therapeutic effect. A gene expression-modifying agent is identified as an agent that is more likely to produce a therapeutic effect on the subject when deviation of the post-treatment genome-wide expression profile from a normal genome-wide expression profile is smaller than deviation of the pre-treatment genome-wide expression profile from the normal genome-wide expression profile, e.g., by at least about 10% or more, including, e.g., at least about 20%, at least about 30%, at least about 40%, at least about 50%, at least about 60%, at least about 70%, at least about 80%, at least about 90% or more.

In contrast, a gene expression-modifying agent is identified as an agent that is less likely to produce a therapeutic effect on the subject when deviation of the post-treatment genome-wide expression profile from a normal genome-wide expression profile is greater than or substantially same as deviation of the pre-treatment genome-wide expression profile from the normal genome-wide expression profile, for example, by at least 10% or more, including, e.g., at least 20%, at least 30%, at least 40%, at least 50%, at least 60%, at least 70%, at least 80%, at least 90%, or more. In some embodiments, a gene expression-modifying agent is identified as an agent that is less likely to produce a therapeutic effect on the subject when deviation of the post-treatment genome-wide expression profile from a normal genome-wide expression profile is greater than or substantially same as deviation of the pre-treatment genome-wide expression profile from the normal genome-wide expression profile, for example, by at least 1.1-fold or more, including, e.g., at least 2-fold, at least 3-fold, at least 4-fold, at least 5-fold, at least 6-fold, at least 7-fold, at least 8-fold, at least 9-fold, at least 10-fold, at least 100-fold, or more.

During the drug selection process, the predicted therapeutic effect of the gene expression-modifying agents in the library that have been selected as agents that are more likely to produce a therapeutic effect can be ranked based on the degree of deviation of the post-treatment genome-wide expression profile from the normal genome-wide expression profile. The smaller the deviation from the normal genome-wide expression profile is, the greater the predicted therapeutic effect the gene-expression-modifying agent produces. Accordingly, in some embodiments, the treatment to be administered to a subject with a disease or disorder can comprise at least one gene expression-modifying agent that yields, among others in the library, the smallest deviation from a normal genome-wide expression profile.

In some embodiments, the treatment can comprise a combination treatment. In some embodiments, the combination treatment can comprise at least two gene expression-modifying agents that are identified to be more likely to produce a therapeutic effect. In some embodiments, the two gene expression-modifying agents can yield, among others in the library, the first two smallest deviations from a normal genome-wide expression profile. Alternatively, the combination of the two gene expression-modifying agents, as an integral treatment, yields, among others in the library, the smallest deviation from a normal genome-wide expression profile.

As used herein, the term “normal genome-wide expression profile” refers to a dataset representing mRNA or transcript levels of a subset of a genome or transcriptome of cells derived from one or a group of (e.g., at least two or more) normal healthy subjects, or derived from the average of a group of (e.g., at least two or more) normal healthy subjects. For example, in some embodiments, a normal genome-wide expression profile can be a dataset representing mRNA or transcript levels of at least about 30% or more, including, e.g., at least about 40%, at least about 50%, at least about 60%, at least about 70%, at least about 80%, at least about 90%, at least about 93%, at least about 95%, at least about 98%, at least about 99%, or more, including 100%, of a genome or transcriptome of cells derived from one or a group of (e.g., at least two or more) normal healthy subjects, or derived from the average of a group of (e.g., at least two or more) normal healthy subjects. In some embodiments where a 2-dimensional PCA plot is used to represent genome-wide expression profiles, a normal genome-wide expression profile can refer to a cluster of loci on the PCA plot, wherein each locus represents a genome-wide expression profile of a normal healthy subject.

The term “normal healthy subject” as used herein refers to a subject who has no symptoms of any diseases or disorders, or who is not identified with any diseases or disorders, or who is not on any medication treatment, or a subject who is identified as healthy by physicians based on medical examinations.

In some embodiments, the gene expression-modifying agent included in the treatment is not clinically known to be indicated for treatment of the disease or disorder that the subject is diagnosed with.

Another aspect relates to a method of identifying a subject who is diagnosed with a disease or disorder and is more likely to respond to a treatment. The method comprises:

(i) assaying a sample from the subject to determine a genome-wide expression profile of the subject;
(ii) in a specifically-programmed computer, computing an expected post-treatment genome-wide expression profile of the subject as a function of the pre-treatment genome-wide expression profile of the subject and known effects of the treatment on gene expression in cells; and
(iii) identifying the subject to be more likely to respond to the treatment when deviation of the post-treatment genome-wide expression profile from a normal genome-wide expression profile is smaller than deviation of the pre-treatment genome-wide expression profile from the normal genome-wide expression profile; or
identifying the subject to be likely to respond to an alternative treatment when deviation of the post-treatment genome-wide expression profile from a normal genome-wide expression profile is greater than or substantially same as deviation of the pre-treatment genome-wide expression profile from the normal genome-wide expression profile.

As used herein, the term “more likely to respond to a treatment” refers to likelihood of a subject to show therapeutic effects.

The subject is identified to be more likely to respond to the treatment when deviation of the post-treatment genome-wide expression profile from a normal genome-wide expression profile is smaller than deviation of the pre-treatment genome-wide expression profile from the normal genome-wide expression profile, e.g., by at least about 10% or more, including, e.g., at least about 20%, at least about 30%, at least about 40%, at least about 50%, at least about 60%, at least about 70%, at least about 80%, at least about 90% or more.

In contrast, the subject is identified to be less likely to respond to the treatment and/or is more likely to benefit from an alternative treatment when deviation of the post-treatment genome-wide expression profile from a normal genome-wide expression profile is greater than or substantially same as deviation of the pre-treatment genome-wide expression profile from the normal genome-wide expression profile, for example, by at least 10% or more, including, e.g., at least 20%, at least 30%, at least 40%, at least 50%, at least 60%, at least 70%, at least 80%, at least 90%, or more. In some embodiments, the subject is identified to be less likely to respond to the treatment and/or is more likely to benefit from an alternative treatment when deviation of the post-treatment genome-wide expression profile from a normal genome-wide expression profile is greater than or substantially same as deviation of the pre-treatment genome-wide expression profile from the normal genome-wide expression profile, for example, by at least 1.1-fold or more, including, e.g., at least 2-fold, at least 3-fold, at least 4-fold, at least 5-fold, at least 6-fold, at least 7-fold, at least 8-fold, at least 9-fold, at least 10-fold, at least 100-fold, or more.

A further aspect relates to a method of drug repositioning. Drug repositioning is the application of known drugs and small molecules to new indications (e.g., new diseases or disorder). The method comprises:

(i) obtaining individual genome-wide expression profiles of patients identified with the same disease or disorder;
(ii) in a specifically-programmed computer, for each identified patient, computing an expected post-treatment genome-wide expression profile of the identified patient as a function of the corresponding individual genome-wide expression profile and known effects of a therapeutic agent on gene expression, wherein the therapeutic agent is not clinically known to be indicated for treatment of the disease or disorder identified in the patients; and
(iii) identifying the therapeutic agent as an agent that is likely to produce a therapeutic effect on the disease or disorder identified in the patients when at least 50% or more of the patients show the expected post-treatment genome-wide expression profile with a smaller deviation from a normal genome-wide expression profile than that of the individual genome-wide expression profile from the normal genome-wide expression profile, thereby computationally repositioning the therapeutic agent for a new indication; or
identifying the therapeutic as an agent that is not likely to produce a therapeutic effect on the disease or disorder identified in the patients when less than 50% of the patients show the expected post-treatment genome-wide expression profile with a smaller deviation from a normal genome-wide expression profile than that of the individual genome-wide expression profile from the normal genome-wide expression profile.

The therapeutic agent is identified as an agent that is likely to produce a therapeutic effect on the disease or disorder identified in the patients when at least 50% or more (including, e.g., at least 60%, at least 70%, at least 80%, at least 90%, at least 95% or more) of the patients show the expected post-treatment genome-wide expression profile with a smaller deviation from a normal genome-wide expression profile than that of the individual genome-wide expression profile from the normal genome-wide expression profile. In these embodiments, the deviation of the expected post-treatment genome-wide expression profile from a normal genome-wide expression profile is smaller than deviation of the individual genome-wide expression profile from the normal genome-wide expression profile, e.g., by at least about 10% or more, including, e.g., at least about 20%, at least about 30%, at least about 40%, at least about 50%, at least about 60%, at least about 70%, at least about 80%, at least about 90% or more.

In contrast, the therapeutic agent is identified as an agent that is not likely to produce a therapeutic effect on the disease or disorder identified in the patients when less than 50% (including, e.g., less than 40%, less than 30%, less than 20%, less than 10%, less than 5% or lower) of the patients show the expected post-treatment genome-wide expression profile with a smaller deviation from a normal genome-wide expression profile than that of the individual genome-wide expression profile from the normal genome-wide expression profile. In these embodiments, the deviation of the expected post-treatment genome-wide expression profile from a normal genome-wide expression profile is smaller than deviation of the individual genome-wide expression profile from the normal genome-wide expression profile, e.g., by at least about 10% or more, including, e.g., at least about 20%, at least about 30%, at least about 40%, at least about 50%, at least about 60%, at least about 70%, at least about 80%, at least about 90% or more.

In some embodiments, the method can further comprise, when the therapeutic agent is computationally repositioned for a new indication, contacting cells in vitro or in an animal model with the therapeutic agent to experimentally validate its therapeutic effect for the new indication. The cells in vitro or in animal model correspond to a model of the same disease or disorder as identified in the patients.

Another aspect relates to a method of identifying a potential adverse effect of a treatment in a subject with a disease or disorder. The method comprises:

(i) assaying a sample from the subject to determine a genome-wide expression profile of the subject;
(ii) in a specifically-programmed computer, computing an expected post-treatment genome-wide expression profile of the subject as a function of the pre-treatment genome-wide expression profile of the subject and known effects of the treatment on gene expression in cells; and
(iii) identifying the treatment to be more likely to induce an adverse effect in the subject when deviation of the post-treatment genome-wide expression profile from a normal genome-wide expression profile is larger than deviation of the pre-treatment genome-wide expression profile from the normal genome-wide expression profile, and/or the post-treatment genome-wide expression profile of the subject is similar to post-treatment genome-wide expression profiles of patients who have suffered from at least one adverse effect upon administration of the same treatment; or
identifying the treatment to be less likely to induce an adverse effect when deviation of the post-treatment genome-wide expression profile from a normal genome-wide expression profile is smaller than deviation of the pre-treatment genome-wide expression profile from the normal genome-wide expression profile, and/or the post-treatment genome-wide expression profile of the subject is different from post-treatment genome-wide expression profiles of patients who have suffered from at least one adverse effect upon administration of the same treatment.

As used herein, the term “adverse effect of a treatment” refers to any undesirable or unfavorable symptoms generated in a subject induced by the administration of the treatment. Examples of adverse effect of a treatment include, but are not limited to, accelerating the progression or severity of a condition associated with a disease or disorder; worsening at least one or more symptoms of a condition, disease or disorder; increasing levels of one or more clinical markers associated with a condition, disease, or disorder; triggering a complication, including, e.g., but not limited to inducing another disease or disorder, tumorigenesis, metastasis, cardiovascular disease, infection, obesity, and any combinations thereof. In some embodiments, the adverse effect of a treatment can include life-threatening symptoms or conditions induced by the treatment. The adverse effect can be chronic or acute.

In some embodiments, the treatment can be identified to be more likely to induce an adverse effect in the subject when deviation of the post-treatment genome-wide expression profile from a normal genome-wide expression profile is larger than deviation of the pre-treatment genome-wide expression profile from the normal genome-wide expression profile, for example, by at least 10% or more, including, e.g., at least 20%, at least 30%, at least 40%, at least 50%, at least 60%, at least 70%, at least 80%, at least 90%, or more. In some embodiments, the treatment can be identified to be more likely to induce an adverse effect in the subject when deviation of the post-treatment genome-wide expression profile from a normal genome-wide expression profile is larger than deviation of the pre-treatment genome-wide expression profile from the normal genome-wide expression profile, for example, by at least 1.1-fold or more, including, e.g., at least 2-fold, at least 3-fold, at least 4-fold, at least 5-fold, at least 6-fold, at least 7-fold, at least 8-fold, at least 9-fold, at least 10-fold, at least 100-fold, or more.

In some embodiments, the treatment is identified to be more likely to induce an adverse effect in the subject when the post-treatment genome-wide expression profile of the subject is similar to (e.g., within 10% or within 5%) reference post-treatment genome-wide expression profiles of patients who have suffered from at least one adverse effect upon administration of the same treatment. In some embodiments, the similarity of the post-treatment genome-wide expression profile of the subject with reference post-treatment genome-wide expression profiles with adverse effect(s) can be represented on a 2-dimensional PCA plot. For example, if the locus of the post-treatment genome-wide expression profile of the subject is within 10% or less for the boundary of a space occupied by loci representing the reference post-treatment genome-wide expression profiles with adverse effect(s), e.g., a heart attack, the treatment is identified to be more likely induce the same or similar type of adverse effect, e.g., heart attack, in the subject if the subject were to be administered with the treatment.

In some embodiments, the treatment is identified to be less likely to induce an adverse effect when deviation of the post-treatment genome-wide expression profile from a normal genome-wide expression profile is smaller than deviation of the pre-treatment genome-wide expression profile from the normal genome-wide expression profile, for example, by at least 10% or more, including, e.g., at least 20%, at least 30%, at least 40%, at least 50%, at least 60%, at least 70%, at least 80%, at least 90%, or more.

In some embodiments, the treatment is identified to be less likely to induce an adverse effect when the post-treatment genome-wide expression profile of the subject deviates (e.g., by at least 10% or more, including, e.g., at least 20%, at last 30%, at least 40%, at least 50%, at least 60%, at least 70%, at least 80%, at least 90%, or more) from reference post-treatment genome-wide expression profiles of patients who have suffered from at least one adverse effect upon administration of the same treatment. In some embodiments, the deviation of the post-treatment genome-wide expression profile of the subject from reference post-treatment genome-wide expression profiles with adverse effect(s) can be represented on a 2-dimensional PCA plot. For example, if the locus of the post-treatment genome-wide expression profile of the subject deviates, e.g., by at least 10% or more, from the boundary of a space A occupied by loci representing the reference post-treatment genome-wide expression profiles with an adverse effect A, e.g., a heart attack, the treatment is identified to be less likely to induce the adverse effect A, e.g., heart attack, in the subject if the subject were to be administered with the treatment.

Accordingly, in some embodiments, the post-treatment genome-wide expression of the subject can be compared to a plurality of reference post-treatment genome-wide expression profiles of patients that demonstrate a plurality of distinct adverse effects. If there is no treatment for the subject that is free of any adverse effect, a treatment that has the least adverse effect can be selected for the subject. Thus, the methods described herein can be used to prioritize treatment options that are available to the subject.

Methods of Representing a Genome-Wide Expression Profile for Computational Analysis:

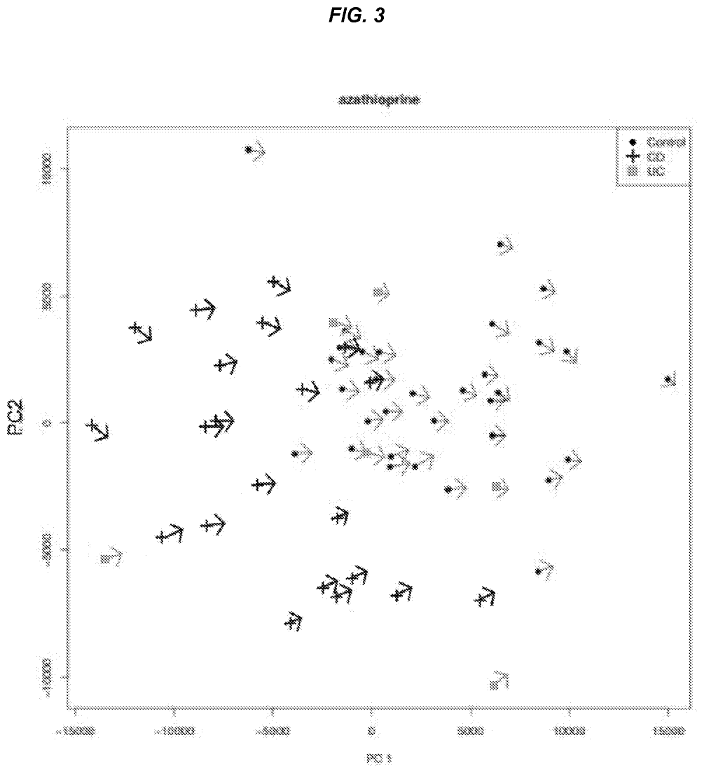

In some embodiments, a genome-wide expression profile can be reduced by mathematical manipulation or transformation, which is explained in detail below, such that it can be represented by 2 or more coordinates, e.g., coordinates determined by PCA as described herein, on a normalized expression plot. By way of example only, as shown in FIGS. 3-5, each locus (shown as a point) on a normalized expression PCA plot represents a genome-wide expression profile of either a diseased subject or a reference subject (e.g., normal healthy subject).

In some embodiments, to construct a normalized gene expression plot, an algorithm comprising principal component analysis can be applied to a compilation of genome-wide expression profiles determined from assayed samples and/or publicly known reference data. The principal component analysis is a mathematical technique known to a skilled artisan for use to compress a multi-dimensional data set by identifying a pattern among components in the data set, followed by transformation of the data to a normalized coordinate system, such that a linear combination of the selected components (e.g., a subset of genes) contributing to the greatest variance of the data set becomes the first principal component (e.g., x-coordinate axis), while the subsequent principal component(s), e.g., the second principal component, can be selected to be orthogonal to the prior principal component (e.g., the first principal component). In some embodiments, the principal component analysis can comprise selecting at least the first two principal components of at least the subset of biochemical expression measurements determined from the reference samples. See, e.g., Abdi H. and Williams L. J. “Principal Component Analysis” Wiley Interdisciplinary Reviews: Computational Statistics. Vol. 2. Issue 4. Page 433-459 (2010) and Lay, David (2000) Linear Algebra and Its Applications. Addision-Wesley, New York; and Kohane I S et al “Microarrays for an Integrative Genomics” Cambridge, Mass., USA: MIT Press (2002), the contents of which are incorporated herein by reference, for information on principal component analysis and how to construct a normalized expression atlas using principal component analysis as well as projection of new data onto the principal components.

Accordingly, in some embodiments of various aspects described herein, the computation involved in the methods can comprise principal component analysis (PCA) of the pre-treatment genome-wide expression profile and the normal genome-wide expression profile to identify a set of gene signatures that are associated with the disease or disorder identified in the subject.

Methods for computing an expected post-treatment genome-wide expression profile: While the expected post-treatment genome-wide expression profile of the subject can be computed as a linear or non-linear function, in some embodiments, the expected post-treatment genome-wide expression profile of the subject can be computed using the following equation:

g_{new}=g+g·dⱼ

wherein g is a gene expression vector or matrix reflecting at least a subset of genes of the pre-treatment genome-wide expression profile of the subject, wherein the subset of genes correspond to the gene signatures associated with the disease or disorder; dⱼ is a transformation matrix reflecting the known modulation of gene expression in cells associated with each of the gene expression-modifying agents; and g_{new} is a gene expression matrix reflecting an expected post-treatment genome-wide expression profile for each of the gene expression-modifying agents.

The known effects of the agent(s) (e.g., drug(s)) on gene expressions in cells can be obtained through in vitro and/or cell-based assays and/or from publicly available in vitro and/or in vivo data, including, e.g., but not limited to, Connectivity Map (CMap). CMap is a catalog of gene-expression data collected from human cells treated with chemical compounds and genetic reagents.

In some embodiments, a statistical model can be used to extract “transcriptional fingerprints” from large gene expression data sets. The model can correct for experiment specific effects (e.g., cell line, batch effects, etc.), and produce an estimate of effect of a drug on a transcriptional profile, dⱼ. This method differs from existing techniques in at least several ways. For example, it can provide continuous effect estimates (e.g., increase in expression of a gene by 20%) instead of binary (up vs. down). It can also combine information across samples to correct for the effects of cell lines to pool all of the information that is available. The method works by estimating experimental conditions using a predicative model (e.g. linear, generalized linear, etc.). The unwanted effects (such as expression changes due to different cell lines, different batches, etc) can then be subtracted to normalize the data.

In some embodiments, the known effects (e.g., in vitro assay measurements and/or publicly available data) of the agent(s) (e.g., drug(s)) on gene expression in cells can be normalized, for example, to improve inter-assay and/or cross-data series comparability. Various art-recognized normalization methods can be used to normalize the known effects of the drug(s) on gene expression in cells. In one embodiment, the normalization method can comprise rank normalization. Other possibly less robust techniques include normalization by the subtracting mean expression value for each gene and dividing by the standard deviation. In another embodiment, prior art methods such as Robust Microarray Average or MASS normalization methods can be used.

Reference genome-wide expression profiles: A reference genome-wide expression profile is a known genome-wide expression profile that is used for comparison with a post-treatment genome-wide expression profile of a subject. Different types of reference genome-wide expression profiles can be used to suit the need to various applications. For example, when one wants to identify a treatment for a subject with a disease or disorder, normal genome-wide expression profiles can be used as reference genome-wide expression profiles for comparison with a post-treatment genome-wide expression profile of a subject. On the other hand, when one wants to identify a potential adverse effect of a treatment, known post-treatment genome-wide expression profiles of subjects who have suffered from at least one or more adverse effect upon administration of the same treatment can be included as reference genome-wide expression profiles.

Reference genome-wide expression profiles are publicly available. For example, Gene Expression Omnibus (GEO) database has over 1.4 million samples.

Inter-assay variations between different sets of the public data are accounted for by rank normalization and other normalization techniques, which allow for different data sets to be comparable, such that they can be passed into a single database for computation.

Methods for comparing a post-treatment genome-wide expression profile of a subject with reference genome-wide expression profiles to measure the deviation: Pre- and post-treatments are compared on the basis of change in correlation and/or distance to the healthy control gene expression profiles. To measure how effective a drug is for treating a specific disease type, the mean correlation pre- and post-treatment between diseased and healthy gene expression profiles can be computed. To compare the treatment benefit for an individual, the predicted pre- and post-treatment correlations can be compared between the individual's gene expression profile and the mean gene expression profile for the healthy control group.

The methods of various aspects described herein can be applied to any disease or disorder. For example, in one embodiment, the methods described herein can be applied to an inflammatory bowel disease. Other disease areas include transcriptionaly mediated diseases including cancers, autoimmune and inflammatory diseases, chronic infections, neuropsychiatric disorders, epilepsy, insulin resistance, developmental delays, and environmental insults. The above list of diseases is merely referred to as an exemplary embodiment and other diseases not mentioned in the list above may also be included.

### Exemplary Embodiment of Systems

Embodiments of a further aspect also provide for systems (and non-transitory computer readable media for causing computer systems) to, e.g., select a treatment for a subject with a disease or disorder and/or to perform the methods of various aspects described herein.

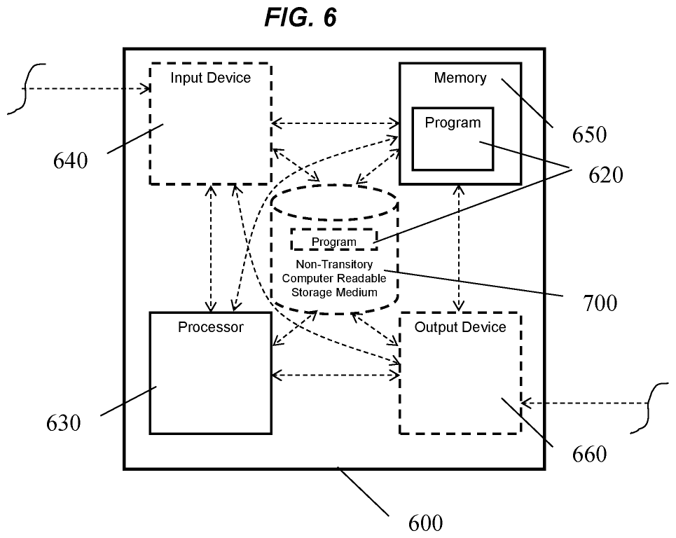

FIG. 6 depicts a device or a computer system 600 comprising one or more processors 630 and a memory 650 storing one or more programs 620 for execution by the one or more processors 630.

In some embodiments, the device or computer system 600 can further comprise a non-transitory computer-readable storage medium 700 storing the one or more programs 620 for execution by the one or more processors 630 of the device or computer system 600.

In some embodiments, the device or computer system 600 can further comprise one or more input devices 640, which can be configured to send or receive information to or from any one from the group consisting of: an external device (not shown), the one or more processors 630, the memory 650, the non-transitory computer-readable storage medium 700, and one or more output devices 660.

In some embodiments, the device or computer system 600 can further comprise one or more output devices 660, which can be configured to send or receive information to or from any one from the group consisting of: an external device (not shown), the one or more processors 630, the memory 650, and the non-transitory computer-readable storage medium 700.

In some embodiments, the device or computer system 600 for performing one of the methods described herein, e.g., selecting a treatment for a subject with a disease or disorder comprises:

one or more processors; and

memory to store one or more programs, the one or more programs comprising instructions for:

(i) computing, for each of a library of gene-expression-modifying agents, an expected post-treatment genome-wide expression profile of the subject as a function of the pre-treatment genome-wide expression profile of the subject and known effects of the corresponding gene expression-modifying agent on gene expression in cells; and

(ii) determining deviation of the post-treatment genome-wide expression profile from a normal genome-wide expression profile;

(iii) determining deviation of the pre-treatment genome-wide expression profile from the normal genome-wide expression profile;

(iv) comparing the deviation of (ii) with the deviation of (iii); and

(iii) displaying a content based in part on the comparison from (iv), wherein the content comprises a signal indicative of the presence of a treatment that is more likely to produce a therapeutic effect on the subject, or a signal indicative of the absence of a treatment that is more likely to produce a therapeutic effect on the subject.

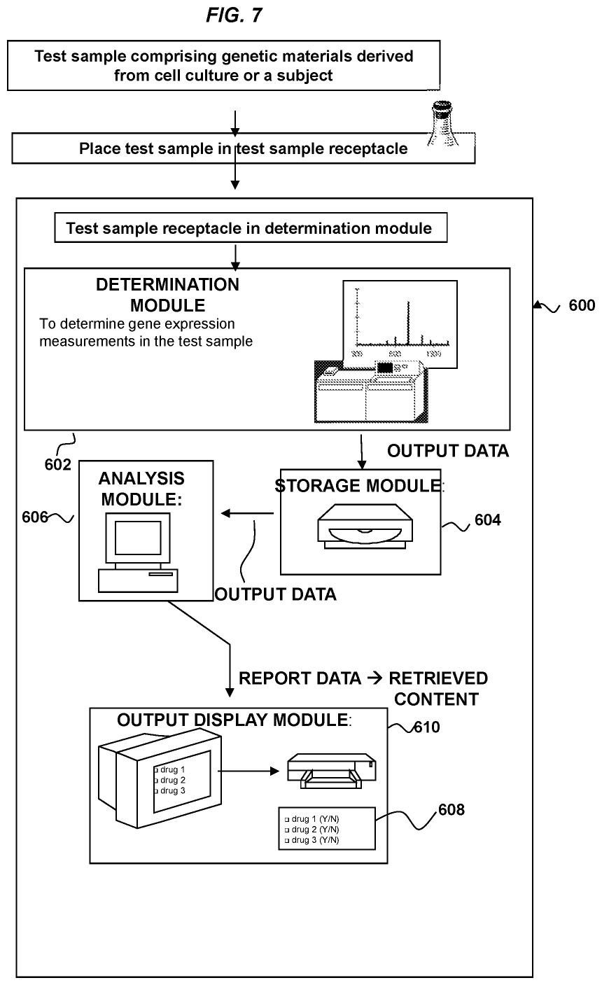

FIG. 7 depicts a device or a system 600 (e.g., a computer system) for obtaining data from at least one test sample comprising genetic materials derived from a cell culture or at least one subject. After placing a test sample in a test sample receptacle and placing the test sample receptacle in a determination module 602, the system can be used for selecting a treatment for a subject. The system comprises:
(a) at least one determination module 602 configured to receive said at least one test sample and perform at least one assay on said at least one test sample comprising a target cell to determine pre-treatment genome-wide expression profile of the subject;
(b) at least one storage device 604 configured to store the pre-treatment genome-wide expression profile of the subject determined from said determination module, and further optionally configured to provide a normalized expression plot reflecting a plurality of reference genome-wide expression profiles described herein,
(c) at least one analysis module 606 configured to perform the following:
   (i) computing, for each of a library of gene-expression-modifying agents, an expected post-treatment genome-wide expression profile of the subject as a function of the pre-treatment genome-wide expression profile of the subject and known effects of the corresponding gene expression-modifying agent on gene expression in cells; and
   (ii) determining deviation of the post-treatment genome-wide expression profile from a normal genome-wide expression profile;
   (iii) determining deviation of the pre-treatment genome-wide expression profile from the normal genome-wide expression profile;
   (iv) comparing the deviation of (ii) with the deviation of (iii); and
(d) at least one display module 610 for displaying a content based in part on the comparison analysis output from said analysis module, wherein the content comprises a signal indicative of the presence of a treatment that is more likely to produce a therapeutic effect on the subject, or a signal indicative of the absence of a treatment that is more likely to produce a therapeutic effect on the subject.

In some embodiments, said at least one determination module 602 can be configured to perform at least one assay selected for determination of gene expression profiling measurements. Various assays for determination of gene expression profiling are known in the art, and can include, e.g., but not limited to, polymerase chain reaction (PCR), real-time quantitative PCR, microarray, RNA sequencing, nucleic acid sequencing, or any combinations thereof. Techniques for nucleic acid sequencing are known in the art and can be used to assay the test sample to determine nucleic acid or gene expression measurements, for example, but not limited to, DNA sequencing, RNA sequencing, de novo sequencing, next-generation sequencing such as massively parallel signature sequencing (MPSS), polony sequencing, pyrosequencing, Illumina (Solexa) sequencing, SOLiD sequencing, ion semiconductor sequencing, DNA nanoball sequencing, Heliscope single molecule sequencing, single molecule real time (SMRT) sequencing), nanopore DNA sequencing, sequencing by hybridization, sequencing with mass spectrometry, microfluidic Sanger sequencing, microscopy-based sequencing techniques, RNA polymerase (RNAP) sequencing, or any combinations thereof.

Depending on the nature of test samples and/or applications of the systems as desired by users, the display module 610 can further display additional content. In some embodiments where the test sample is collected or derived from a subject for diagnostic assessment, the content displayed on the display module 610 can further comprise a signal indicative of a diagnosis of a condition (e.g., disease or disorder) or a state of the condition (e.g., disease or disorder) in the subject.

In some embodiments wherein the test sample is collected or derived from a subject for selection and/or evaluation of a treatment regimen for a subject, the content can further comprise a signal indicative of a treatment regimen personalized to the subject, based on the magnitude of the deviation of the expected post-treatment genome-wide expression profile from a normal genome-wide expression profile.

In some embodiments, the at least one analysis module 606 can be configured to determine trajectory of the locus corresponding to an expected post-treatment gene expression of a subject, e.g., by comparing the current locus with its previously-determined locus. Thus, the progression of a condition (e.g., a disease or disorder), and/or the effectiveness of a treatment regimen administered to a subject with the condition can be determined.

A tangible and non-transitory (e.g., no transitory forms of signal transmission) computer readable medium 700 having computer readable instructions recorded thereon to define software modules for implementing a method on a computer is also provided herein. In some embodiments, the computer readable medium 700 stores one or more programs for performing one of the methods described herein, e.g., for selecting a treatment for a subject with a disease or disorder. The one or more programs for execution by one or more processors of a computer system comprises (a) instructions for analyzing the data (e.g., pre-treatment genome-wide expression profile of one or more subjects) stored on a storage device, wherein the analyzing comprises the following: (i) computing, for each of a library of gene-expression-modifying agents, an expected post-treatment genome-wide expression profile of the subject as a function of the pre-treatment genome-wide expression profile of the subject and known effects of the corresponding gene expression-modifying agent on gene expression in cells; and (ii) determining deviation of the post-treatment genome-wide expression profile from a normal genome-wide expression profile; (iii) determining deviation of the pre-treatment genome-wide expression profile from the normal genome-wide expression profile; (iv) comparing the deviation of (ii) with the deviation of (iii); and (b) instructions for displaying a content based in part on the data output from the analysis module, wherein the content comprises a signal indicative of the presence of a treatment that is more likely to produce a therapeutic effect on the subject, or a signal indicative of the absence of a treatment that is more likely to produce a therapeutic effect on the subject.

Depending on the nature of test samples and/or applications of the systems as desired by users, the computer readable storage medium 700 can further comprise instructions for displaying additional content. In some embodiments where the test sample is collected or derived from a subject for diagnostic assessment, the content displayed on the display module can further comprise a signal indicative of a diagnosis of a condition (e.g., disease or disorder) or a state of the condition (e.g., disease or disorder) in the subject. For example, in some embodiments wherein the test sample is collected or derived from a subject for selection and/or evaluation of a treatment regimen for a subject, the content can further comprise a signal indicative of a treatment regimen personalized to the subject, based in part on the deviation of the expected post-treatment genome-wide expression profile from a normal genome-wide expression profile.

In some embodiments, the instructions for the analyzing can further comprise determining trajectory of the locus corresponding to an expected post-treatment gene expression of a subject, e.g., by comparing the current locus with its previously-determined locus. Thus, the progression of a condition (e.g., a disease or disorder), and/or the effectiveness of a treatment regimen administered to a subject with the condition can be determined.

Embodiments of the systems described herein have been described through functional modules, which are defined by computer executable instructions recorded on computer readable media and which cause a computer to perform method steps when executed. The modules have been segregated by function for the sake of clarity. However, it should be understood that the modules need not correspond to discrete blocks of code and the described functions can be carried out by the execution of various code portions stored on various media and executed at various times. Furthermore, it should be appreciated that the modules may perform other functions, thus the modules are not limited to having any particular functions or set of functions.

Computing devices typically include a variety of media, which can include computer-readable storage media and/or communications media, in which these two terms are used herein differently from one another as follows. Computer-readable storage media or computer readable media (e.g., 700) can be any available tangible media (e.g., tangible storage media) that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM (random access memory), ROM (read only memory), EEPROM (erasable programmable read only memory), flash memory or other memory technology, CD-ROM (compact disc read only memory), DVD (digital versatile disk) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In some embodiments, the computer readable storage media 700 can include the “cloud” system, in which a user can store data on a remote server, and later access the data or perform further analysis of the data from the remote server.

Computer-readable data embodied on one or more computer-readable media, or computer readable medium 700, may define instructions, for example, as part of one or more programs, that, as a result of being executed by a computer, instruct the computer to perform one or more of the functions described herein (e.g., in relation to system 600, or computer readable medium 700), and/or various embodiments, variations and combinations thereof. Such instructions may be written in any of a plurality of programming languages, for example, Java, J #, Visual Basic, C, C #, C++, Fortran, Pascal, Eiffel, Basic, COBOL assembly language, and the like, or any of a variety of combinations thereof. The computer-readable media on which such instructions are embodied may reside on one or more of the components of either of system 600, or computer readable medium 700 described herein, may be distributed across one or more of such components, and may be in transition there between.

The computer-readable media can be transportable such that the instructions stored thereon can be loaded onto any computer resource to implement the assays and/or methods described herein. In addition, it should be appreciated that the instructions stored on the computer readable media, or computer-readable medium 700, described above, are not limited to instructions embodied as part of an application program running on a host computer. Rather, the instructions may be embodied as any type of computer code (e.g., software or microcode) that can be employed to program a computer to implement the methods described herein. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the system or computer system described herein can include a determination module, a storage device, an analysis module and a display module. The functional modules can be executed on one, or multiple, computers, or by using one, or multiple, computer networks. The determination module 602 can have computer executable instructions to perform at least one assay selected for determination of gene expression profiles of at least one or more subjects.

In some embodiments, the determination module 602 can have computer executable instructions to provide sequence information in computer readable form, e.g., for RNA sequencing. As used herein, “sequence information” refers to any nucleotide and/or amino acid sequence, including but not limited to full-length nucleotide and/or amino acid sequences, partial nucleotide and/or amino acid sequences, or mutated sequences. Moreover, information “related to” the sequence information includes detection of the presence or absence of a sequence (e.g., detection of a mutation or deletion), determination of the concentration of a sequence in the sample (e.g., amino acid sequence expression levels, or nucleotide (RNA or DNA) expression levels), and the like. The term “sequence information” is intended to include the presence or absence of post-translational modifications (e.g. phosphorylation, glycosylation, summylation, farnesylation, and the like).

As an example, determination modules 602 for determining sequence information may include known systems for automated sequence analysis including but not limited to Hitachi FMBIO® and Hitachi FMBIO® II Fluorescent Scanners (available from Hitachi Genetic Systems, Alameda, Calif.); Spectrumedix® SCE 9610 Fully Automated 96-Capillary Electrophoresis Genetic Analysis Systems (available from SpectruMedix LLC, State College, Pa.); ABI PRISM® 377 DNA Sequencer, ABI® 373 DNA Sequencer, ABI PRISM® 310 Genetic Analyzer, ABI PRISM® 3100 Genetic Analyzer, and ABI PRISM® 3700 DNA Analyzer (available from Applied Biosystems, Foster City, Calif.); Molecular Dynamics Fluorlmager™ 575, SI Fluorescent Scanners, and Molecular Dynamics Fluorlmager™ 595 Fluorescent Scanners (available from Amersham Biosciences UK Limited, Little Chalfont, Buckinghamshire, England); GenomyxSC™ DNA Sequencing System (available from Genomyx Corporation (Foster City, Calif.); and Pharmacia ALF™ DNA Sequencer and Pharmacia ALFexpress™ (available from Amersham Biosciences UK Limited, Little Chalfont, Buckinghamshire, England).

Alternative methods for determining sequence information, i.e. determination modules 602, include systems for protein and DNA analysis. For example, mass spectrometry systems including Matrix Assisted Laser Desorption Ionization-Time of Flight (MALDI-TOF) systems and SELDI-TOF-MS ProteinChip array profiling systems; systems for analyzing gene expression data (see, for example, published U.S. Patent Application Pub. No. U.S. 2003/0194711); systems for array based expression analysis: e.g., HT array systems and cartridge array systems such as GeneChip® AutoLoader, Complete GeneChip® Instrument System, GeneChip® Fluidics Station 450, GeneChip® Hybridization Oven 645, GeneChip® QC Toolbox Software Kit, GeneChip® Scanner 3000 7G plus Targeted Genotyping System, GeneChip® Scanner 3000 7G Whole-Genome Association System, GeneTitan™ Instrument, and GeneChip® Array Station (each available from Affymetrix, Santa Clara, Calif.); automated ELISA systems (e.g., DSX® or DS2® (available from Dynax, Chantilly, Va.) or the Triturus® (available from Grifols USA, Los Angeles, Calif.), The Mago® Plus (available from Diamedix Corporation, Miami, Fla.); Densitometers (e.g. X-Rite-508-Spectro Densitometer® (available from RP Imaging™, Tucson, Ariz.), The HYRYS™ 2 HIT densitometer (available from Sebia Electrophoresis, Norcross, Ga.); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, (available from Becton Dickinson, Franklin Lakes, N.J.); and radio isotope analyzers (e.g. scintillation counters).

The gene expression profiling measurements determined in the determination module can be read by the storage device 604. As used herein the “storage device” 604 is intended to include any suitable computing or processing apparatus or other device configured or adapted for storing data or information. Examples of electronic apparatus suitable for use with the system described herein can include stand-alone computing apparatus, data telecommunications networks, including local area networks (LAN), wide area networks (WAN), Internet, Intranet, and Extranet, and local and distributed computer processing systems. Storage devices 604 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device 604 is adapted or configured for having recorded thereon sequence information or expression level information. Such information may be provided in digital form that can be transmitted and read electronically, e.g., via the Internet, on diskette, via USB (universal serial bus) or via any other suitable mode of communication, e.g., the “cloud”.

As used herein, “expression level information” refers to any nucleic acid (e.g., RNA/DNA), gene, and/or protein or peptide expression measurements. In some embodiments, the expression level information can be determined from the sequence information determined from the determination module. In some embodiments, the expression level information can be determined from a hybridization-based microarray.

As used herein, “stored” refers to a process for encoding information on the storage device 604. Those skilled in the art can readily adopt any of the presently known methods for recording information on known media to generate manufactures comprising the sequence information or expression level information.

A variety of software programs and formats can be used to store the sequence information or expression level information on the storage device. Any number of data processor structuring formats (e.g., text file or database) can be employed to obtain or create a medium having recorded thereon the sequence information or expression level information.

By providing sequence information and/or expression level information in computer-readable form, one can use the sequence information and/or expression level information in readable form (e.g., as a multi-dimensional expression vector) in the analysis module 606 to (i) compute, for each of a library of gene-expression-modifying agents, an expected post-treatment genome-wide expression profile of the subject as a function of the pre-treatment genome-wide expression profile of the subject and known effects of the corresponding gene expression-modifying agent on gene expression in cells; (ii) determine deviation of the post-treatment genome-wide expression profile from a normal genome-wide expression profile; (iii) determine deviation of the pre-treatment genome-wide expression profile from the normal genome-wide expression profile; and (iv) compare the deviation of (ii) with the deviation of (iii).

In some embodiments, the expected post-treatment genome-wide expression profile of the subject can be computed using the following equation:

g_{new}=g+g·d_{g}

wherein g is a gene expression vector reflecting at least a subset of genes of the pre-treatment genome-wide expression profile of the subject, wherein the subset of genes correspond to the gene signatures associated with the disease or disorder; d_{g} is a transformation matrix reflecting the known modulation of gene expression in cells associated with each of the gene expression-modifying agents; and g_{new} is a gene expression matrix reflecting an expected post-treatment genome-wide expression profile for each of the gene expression-modifying agents. The analysis made in computer-readable form provides a computer readable analysis result which can be processed by a variety of means. Content 608 based on the analysis result can be retrieved from the analysis module 606 to indicate the presence or absence of a treatment that is more likely to produce a therapeutic effect on the subject.

In one embodiment, the storage device 604 to be read by the analysis module 606 can comprise expression array datasets that are electronically or digitally recorded and publicly available through public repositories such as National Center for Biotechnology Information (NCBI's) Gene Expression Omnibus (GEO). These expression array datasets can then ready by an analysis module 606 to generate reference genome-wide expression profiles. Additionally, the storage device 604 to be read by the analysis module 606 can comprise expression array datasets that are electronically or digitally recorded and publicly available through public repositories such as Connectivity Map (CMap). These gene-expression data collected from human cells treated with chemical compounds and genetic reagents can be used to generate d_{g} as described above.

The “analysis module” 606 can use a variety of available software programs and formats for construction of the normalized expression plot to represent pre-treatment genome-wide expression profiles, expected pre-treatment genome-wide expression profiles, and/or reference genome-wide expression profiles. In one embodiment, the analysis module 606 can be configured to project the genome-wide expression profiles onto the principle components (e.g., PC1 and PC2) of a normalized expression plot, which is constructed based on principal component analysis. See, e.g., Abdi H. and Williams L. J. “Principal Component Analysis” Wiley Interdisciplinary Reviews: Computational Statistics. Vol. 2. Issue 4. Page 433-459 (2010) and Lay, David (2000) Linear Algebra and Its Applications. Addision-Wesley, New York; and Kohane I S et al “Microarrays for an Integrative Genomics” Cambridge, Mass., USA: MIT Press (2002), for information on principal component analysis and how to construct a normalized expression plot using principal component analysis as well as projection of new data onto the principal components. The analysis module 606 may be configured using existing commercially-available or freely-available software for performing principal component analysis.

In some embodiments, the analysis module 606 can further comprise software programs and/or algorithms (e.g., vector analysis) to determine trajectory of the locus corresponding to an expected post-treatment gene expression of a subject, e.g., by comparing the current locus with its previously-determined locus.

In some embodiments, the analysis module 606 can be configured to perform normalization of gene expression data obtained from public repositories such GEO and/or scientific publications, as well as pre-treatment genome-wide expression profile determined from the determination module 602. Different software and algorithms for data normalization are known in the art. For example, in one embodiment, the analysis module 606 can be configured to normalize the expression data via R's BioConductor package. The resulting probe set intensities are averaged into unique, e.g., gene-centric values, and then rank normalized to improve cross-data series comparability. The calculations can be performed in the R statistical environment, employing the BioConductors suite. See, e.g., R Development Core Team “R: A language and environment for statistical computing.” Vienna, Austria 2007; and Gentleman R C et al. “Bioconductor: open software development for computational biology and bioinformatics.” Genome Biol 2004, 5: R80, for exemplary methods of data normalization.

Various algorithms are available which are useful for comparing multi-dimensional data (e.g., microarray data analysis) and/or identifying the predictive gene signatures. For example, algorithms such as those identified in Babu M. M. “Introduction to microarray data analysis” in Computational Genomics (Ed: R. Grant), Horizon Press, U.K.; Komura et al. “Multidimensional support vector machines for visualization of gene expression data” Bioinformatics Vol. 21 (2005) 439; Montaner D. and Dopazo J. “Multidimensional gene set analysis of genomic data” PLoS One, April 2010 (Vol. 5, Issue 4) e10348; Piro R. M. “An atlas of tissue specific conserved coexpression for functional annotation and disease gene prediction” European Journal of Human Genetics (2011) 19, 1173-1180; Zhang S. et al. “Discovery of multi-dimensional modules by integrative analysis of cancer genomic data” Nucleic acids research 2012 (1-13); Breitling R. et al. “Vector analysis as a fast and easy method to compare gene expression responses between different experimental backgrounds” BMC Bioinformatics 2005, 6: 181; Guo W et al. “Controlling false discoveries in multidimensional directional decisions, with applications to gene expression data on ordered categories.” Biometrics. 2010 June; 66(2):485-92; van Deun K. et al. “Joint mapping of genes and conditions via multidimensional unfolding analysis.” BMC bioinformatics 2007, 8: 181; and Hutz J. E. et al. “The multidimensional perturbation value: A single metric to measure similarity and activity of treatments in high-throughput multidimensional screens.” Journal of Biomolecule screening (published online 20 Nov. 2012), or any combinations thereof can also be used in the analysis module 606.

The analysis module 606, or any other module of the system described herein, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular embodiment, users can directly access data (via Hypertext links for example) residing on Internet databases using a HTML interface provided by Web browsers and Web servers. In another embodiment, users can directly access data residing on the “cloud” provided by the cloud computing service providers.

The analysis module 606 provides computer readable analysis result that can be processed in computer readable form by predefined criteria, or criteria defined by a user, to provide a content based in part on the analysis result that may be stored and output as requested by a user using a display module 610. The display module 610 enables display of a content 608 based in part on the comparison result for the user, wherein the content 608 is a signal indicative of the presence of a treatment that is more likely to produce a therapeutic effect on the subject, or a signal indicative of the absence of a treatment that is more likely to produce a therapeutic effect on the subject. Such signal, can be for example, a display of content 608 indicative of the presence or absence of a treatment that is more likely to produce a therapeutic effect on a computer monitor, a printed page of content 608 indicating the presence or absence of a treatment that is more likely to produce a therapeutic effect from a printer, or a light or sound indicative of the presence or absence of a treatment that is more likely to produce a therapeutic effect.

In various embodiments of the computer system described herein, the analysis module 606 can be integrated into the determination module 602.

Depending on the nature of test samples and/or applications of the systems as desired by users, the content 608 based on the analysis result can also include a signal indicative of a diagnosis of a condition (e.g., disease or disorder) or a state of the condition (e.g., disease or disorder) in the subject.

In some embodiments, the content 608 based on the analysis result can include a graphical representation reflecting the loci (corresponding to the pre-treatment genome-wide expression profile and/or post-treatment genome-wide expression profile) relative to a plurality of reference loci (corresponding to reference genome-wide expression profiles).

In one embodiment, the content 608 based on the analysis result is displayed a on a computer monitor. In one embodiment, the content 608 based on the analysis result is displayed through printable media. The display module 610 can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content 608 based on the analysis result. It should be understood that other modules of the system described herein can be adapted to have a web browser interface. Through the Web browser, a user may construct requests for retrieving data from the analysis module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information related to a treatment regimen for a subject with a disease or disorder, e.g., display of an indication of a treatment that is more likely to produce a therapeutic effect, or display of information based thereon. In one embodiment, the information of the reference genome-wide expression profiles is also displayed.

In any embodiments, the analysis module can be executed by a computer implemented software as discussed earlier. In such embodiments, a result from the analysis module can be displayed on an electronic display. The result can be displayed by graphs, numbers, characters or words. In additional embodiments, the results from the analysis module can be transmitted from one location to at least one other location. For example, the comparison results can be transmitted via any electronic media, e.g., internet, fax, phone, a “cloud” system, and any combinations thereof. Using the “cloud” system, users can store and access personal files and data or perform further analysis on a remote server rather than physically carrying around a storage medium such as a DVD or thumb drive.

Each of the above identified modules or programs corresponds to a set of instructions for performing a function described above. These modules and programs (i.e., sets of instructions) need not be implemented as separate software programs, procedures or modules, and thus various subsets of these modules may be combined or otherwise re-arranged in various embodiments. In some embodiments, memory may store a subset of the modules and data structures identified above. Furthermore, memory may store additional modules and data structures not described above.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described herein can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described herein. Furthermore, not all illustrated acts may be required to implement the methodologies in accordance with the disclosed subject matter. In addition, those skilled in the art will understand and appreciate that the methodologies could alternatively be represented as a series of interrelated states via a state diagram or events. Additionally, it should be appreciated that the methodologies disclosed in this specification are capable of being stored on an article of manufacture to facilitate transporting and transferring such methodologies to computing devices. The term article of manufacture, as used herein, is intended to encompass a computer program accessible from any computer-readable device or storage media.

The system 600, and computer readable medium 700, are merely illustrative embodiments, e.g., for selecting a treatment for a subject with a disease or disorder and/or for use in the methods of various aspects described herein and is not intended to limit the scope of the inventions described herein. Variations of system 600, and computer readable medium 700, are possible and are intended to fall within the scope of the inventions described herein.

The modules of the machine, or used in the computer readable medium, may assume numerous configurations. For example, function may be provided on a single machine or distributed over multiple machines.

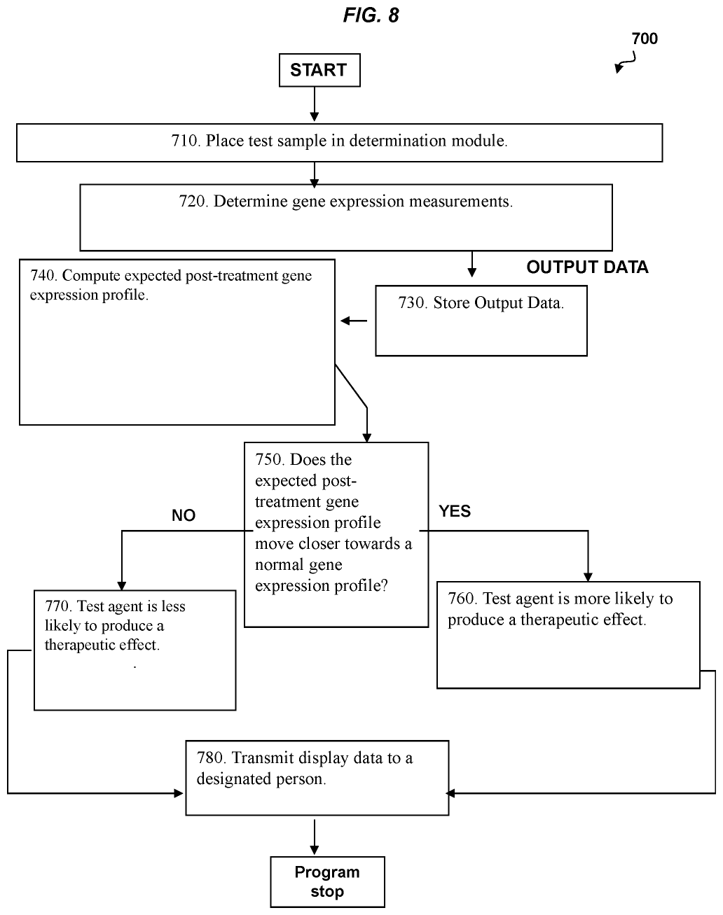

FIG. 8 shows an exemplary methodology 700 with a set of instructions on a computer readable medium for use with a system 600 according to an embodiment of the present disclosure. At step 710, the methodology provides for first placing at least one test sample comprising genetic materials into a determination module.

At step 720, the determination system then determines gene expression measurements in the test sample. The determination system will provide output data at this step.

At step 730, output data from the determination module can be stored on a storage module.

At step 740, an analysis module can compute, for each test agent, an expected post-treatment gene expression profile g_{new} of the test sample, e.g., using the following equation:

g_{new}=g+g·dg.

In this equation, g is pre-treatment gene expression data (output data stored data from determination module) and dg represents effects of the test agent on gene expression.

At step 740, the set of instructions can then check whether g_{new} moves closer than g toward a normal gene expression profile. This can be determined, for example, by distance analysis in an iterative process for each different test agent.

If g_{new} did move closer than g towards a normal gene expression profile, then the display module indicates at step 760 that the test agent is more likely to produce a therapeutic effect on the cell or subject from which the test sample is derived. If g_{new} did not move closer than g towards a normal gene expression profile, then the display module indicates at step 770 that the test agent is less likely to produce a therapeutic effect on the cell or subject from which the test sample is derived.

Regardless of whether the method performs step 760 or step 770 previously, the methodology 700 proceeds to step 780. At step 780, the methodology 700 can then provide for transmitting display data to a designated person, such as the user, a patient, or a physician.

### Sample and Assays

In accordance with embodiments of various aspects described herein, a test sample or sample, including any fluid or specimen (processed or unprocessed) or other biological sample, can be subjected to the methods and systems described herein. The test sample or fluid can be liquid, supercritical fluid, solutions, suspensions, gases, gels, slurries, and combinations thereof. The test sample or fluid can be aqueous or non-aqueous. In some embodiments, the sample or test sample comprises genetic materials, such as nucleic acids (e.g., DNA and/or mRNA), for measurements of gene expression.

In some embodiments, the test sample can include a biological fluid obtained from a subject. Exemplary biological fluids obtained from a subject can include, but are not limited to, blood (including whole blood, plasma, cord blood and serum), lactation products (e.g., milk), amniotic fluids (e.g., a sample collected during amniocentesis), sputum, saliva, urine, semen, cerebrospinal fluid, bronchial aspirate, perspiration, mucus, liquefied feces, synovial fluid, lymphatic fluid, tears, tracheal aspirate, and fractions thereof. In some embodiments, a biological fluid can include a homogenate of a tissue specimen (e.g., biopsy) from a subject. In one embodiment, a test sample can comprise a suspension obtained from homogenization of a solid sample obtained from a solid organ or a fragment thereof.

In some embodiments, a test sample can be obtained from a normal healthy subject. In other embodiments, a test sample can be obtained from a subject who has or is suspected of having a disease or disorder, e.g., a condition afflicting a tissue, or who is suspected of having a risk of developing a disease or disorder, e.g., a condition afflicting a tissue. In some embodiments, the test sample can be obtained from a subject who has or is suspected of having cancer, or who is suspected of having a risk of developing cancer. In some embodiments, the test sample can be obtained from a subject who has or is suspected of having a neurodegenerative disorder, or who is suspected of having a risk of developing neurodegenerative disorder. In some embodiments, the test sample can be obtained from a subject who has or is suspected of having an inflammatory bowel disease.

In some embodiments, a test sample can be obtained from a subject who is being treated for the disease or disorder. In other embodiments, the test sample can be obtained from a subject whose previously-treated disease or disorder is in remission. In other embodiments, the test sample can be obtained from a subject who has a recurrence of a previously-treated disease or disorder. For example, in the case of cancer such as breast cancer or pancreatic cancer, a test sample can be obtained from a subject who is undergoing a cancer treatment, or whose cancer was treated and is in remission, or who has cancer recurrence.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, and avian species, e.g., chicken, emu, ostrich. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female. The term “patient” and “subject” does not denote a particular age. Thus, any mammalian subjects from adult to newborn subjects, as well as fetuses, are intended to be covered.

In one embodiment, the subject or patient is a mammal. The mammal can be a human, non-human primate, mouse, rat, dog, cat, horse, or cow, but are not limited to these examples. In one embodiment, the subject is a human being. In another embodiment, the subject can be a domesticated animal and/or pet.

In some embodiments, the test sample can include a fluid (e.g., culture medium) from a biological culture. Examples of a fluid (e.g., culture medium) obtained from a biological culture includes the one obtained from culturing or fermentation, for example, of single- or multi-cell organisms, including prokaryotes (e.g., bacteria) and eukaryotes (e.g., animal cells, plant cells, insect cells, yeasts, fungi), and including fractions thereof. In some embodiments, the test sample can include a fluid from a blood culture. In some embodiments, the culture medium can be obtained from any source, e.g., without limitations, research laboratories, pharmaceutical manufacturing plants, hydrocultures (e.g., hydroponic food farms), diagnostic testing facilities, clinical settings, and any combinations thereof.

In some embodiments, the test sample can include a media or reagent solution used in a laboratory or clinical setting, such as for biomedical and molecular biology applications. As used herein, the term “media” refers to a medium for maintaining a tissue, an organism, or a cell population, or refers to a medium for culturing a tissue, an organism, or a cell population, which contains nutrients that maintain viability of the tissue, organism, or cell population, and support proliferation and growth.

As used herein, the term “reagent” refers to any solution used in a laboratory or clinical setting for biomedical and molecular biology applications. Reagents include, but are not limited to, saline solutions, PBS solutions, buffered solutions, such as phosphate buffers, EDTA, Tris solutions, and any combinations thereof. Reagent solutions can be used to create other reagent solutions. For example, Tris solutions and EDTA solutions are combined in specific ratios to create “TE” reagents for use in molecular biology applications.

In some embodiments, a sample comprising genetic materials such as DNA and/or mRNA, can be derived from cells or tissues derived from a subject, e.g., mammalian subjects. Exemplary mammalian cells include, but are not limited to, stem cells (e.g., naturally existing stem cells or derived stem cells), cancer cells, progenitor cells, immune cells, blood cells, fetal cells, and any combinations thereof. The cells can be derived from a wide variety of tissue types without limitation such as; hematopoietic, neural, mesenchymal, cutaneous, mucosal, stromal, muscle, spleen, reticuloendothelial, epithelial, endothelial, hepatic, kidney, gastrointestinal, pulmonary, cardiovascular, and T-cells, and fetus. Stem cells, embryonic stem (ES) cells, ES-derived cells, induced pluripotent stem cells, and stem cell progenitors are also included, including without limitation, hematopoietic, neural, stromal, muscle, cardiovascular, hepatic, pulmonary, and gastrointestinal stem cells. In some embodiments, the cells can be ex vivo or cultured cells, e.g. in vitro. For example, for ex vivo cells, cells can be obtained from a subject, where the subject is healthy and/or affected with a disease. While cells can be obtained from a fluid sample, e.g., a blood sample, cells can also be obtained, as a non-limiting example, by biopsy or other surgical means know to those skilled in the art.

In some embodiments, a sample comprising genetic materials such as DNA and/or mRNA can be derived from any cell type or any tissue type from any species (e.g., animal, mammal, plant, insect, and/or microbes). In some embodiments, examples of cell types can include, but are not limited to, somatic cells, stem cells (e.g., naturally existing stem cells or derived stem cells such as iPSCs), germ cells, bone marrow cells, adipose cells, dermal cells, epidermal cells, epithelial cells, connective tissue cells, fibroblasts, muscle cells, cartilage cells, chondrocytes, ocular cells, follicle cells, buccal cells, neuronal cells, reproductive cells, and/or blood cells), or of any tissue type (e.g., but not limited to, lung, liver, colon, heart, skin, brain, gastrointestinal, bone, and/or breast) from a mammalian subject. For example, a mammalian subject can be a human subject.

The sample used to determine a genome-wide expression profile of the subject(s) in the methods of various aspects described herein can be assayed by any methods known in the art. For example, the sample can be assayed by a method comprising polymerase chain reaction (PCR), a real-time quantitative PCR, microarray, RNA sequencing, and/or nucleic acid sequencing.

Conditions (e.g., Diseases or Disorders) Amenable to Diagnosis, Prognosis/Monitoring, and/or Treatment Using Methods, Systems or Various Aspects Described Herein

Different embodiments of the methods and systems described herein can be used for diagnosis and/or treatment of a disease or disorder, and/or the state of the disease or disorder in a subject, e.g., a condition afflicting a certain tissue in a subject. For example, the disease or disorder in a subject can be associated with breast, pancreas, blood, prostate, colon, lung, skin, brain, ovary, kidney, oral cavity, throat, cerebrospinal fluid, liver, or other tissues, and any combination thereof.

In some embodiments, the condition (e.g., disease or disorder) amenable to diagnosis and/or treatment using any aspects described herein can include a condition that is not terminal but can cause an interruption, disturbance, or cessation of a bodily function, system, or organ. Such examples of disorders can include, e.g., but not limited to, developmental disorders (e.g., autism), brain disorders (e.g., epilepsy), mental disorders (e.g., depression), endocrine disorders (e.g., diabetes), or skin disorders (e.g., skin inflammation).

In some embodiments, the condition (e.g., disease or disorder) amenable to diagnosis and/or treatment using any aspects described herein can include a breast disease or disorder. Exemplary breast disease or disorder includes breast cancer.

In some embodiments, the condition (e.g., disease or disorder) amenable to diagnosis and/or treatment using any aspects described herein can include a pancreatic disease or disorder. Non-limiting examples of pancreatic diseases or disorders include acute pancreatitis, chronic pancreatitis, hereditary pancreatitis, pancreatic cancer (e.g., endocrine or exocrine tumors), etc., and any combinations thereof.

In some embodiments, the condition (e.g., disease or disorder) amenable to diagnosis and/or treatment using any aspects described herein can include a blood disease or disorder. Examples of blood disease or disorder include, but are not limited to, platelet disorders, von Willebrand diseases, deep vein thrombosis, pulmonary embolism, sickle cell anemia, thalassemia, anemia, aplastic anemia, fanconi anemia, hemochromatosis, hemolytic anemia, hemophilia, idiopathic thrombocytopenic purpura, iron deficiency anemia, pernicious anemia, polycythemia vera, thrombocythemia and thrombocytosis, thrombocytopenia, and any combinations thereof.

In some embodiments, the condition (e.g., disease or disorder) amenable to diagnosis and/or treatment using any aspects described herein can include a prostate disease or disorder. Non-limiting examples of a prostate disease or disorder can include prostatis, prostatic hyperplasia, prostate cancer, and any combinations thereof.

In some embodiments, the condition (e.g., disease or disorder) amenable to diagnosis and/or treatment using any aspects described herein can include a colon disease or disorder. Exemplary colon diseases or disorders can include, but are not limited to, colorectal cancer, colonic polyps, ulcerative colitis, diverticulitis, and any combinations thereof.

In some embodiments, the condition (e.g., disease or disorder) amenable to diagnosis and/or treatment using any aspects described herein can include a lung disease or disorder. Examples of lung diseases or disorders can include, but are not limited to, asthma, chronic obstructive pulmonary disease, infections, e.g., influenza, pneumonia and tuberculosis, and lung cancer.

In some embodiments, the condition (e.g., disease or disorder) amenable to diagnosis and/or treatment using any aspects described herein can include a skin disease or disorder, or a skin condition. An exemplary skin disease or disorder can include skin cancer.

In some embodiments, the condition (e.g., disease or disorder) amenable to diagnosis and/or treatment using any aspects described herein can include a brain or mental disease or disorder (or neural disease or disorder). Examples of brain diseases or disorders (or neural disease or disorder) can include, but are not limited to, brain infections (e.g., meningitis, encephalitis, brain abscess), brain tumor, glioblastoma, stroke, ischemic stroke, multiple sclerosis (MS), vasculitis, and neurodegenerative disorders (e.g., Parkinson's disease, Huntington's disease, Pick's disease, amyotrophic lateral sclerosis (ALS), dementia, and Alzheimer's disease), Timothy syndrome, Rett syndrome, Fragile X, autism, schizophrenia, spinal muscular atrophy, frontotemporal dementia, any combinations thereof.

In some embodiments, the condition (e.g., disease or disorder) amenable to diagnosis and/or treatment using any aspects described herein can include a liver disease or disorder. Examples of liver diseases or disorders can include, but are not limited to, hepatitis, cirrhosis, liver cancer, billary cirrhosis, primary sclerosing cholangitis, Budd-Chiari syndrome, hemochromatosis, transthyretin-related hereditary amyloidosis, Gilbert's syndrome, and any combinations thereof.

In some embodiments, the condition (e.g., disease or disorder) amenable to diagnosis and/or treatment using any aspects described herein can include an inflammatory bowel disease. Examples of inflammatory bowel disease can include, but are not limited to, ulcerative colitis and Crohn's disease.

In other embodiments, the condition (e.g., disease or disorder) amenable to diagnosis and/or treatment using any aspects described herein can include cancer. Examples of cancers can include, but are not limited to, bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS associated leukemias and adult T-cell leukemia lymphoma; intraepithelial neoplasms including Bowen's disease and Paget's disease, liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilm's tumor.

### Some Selected Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with numeric values means±5%.

In one aspect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

The words “example” or “exemplary” or “e.g.,” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

As used herein, the term “a plurality of” refers to at least 2 or more, including, e.g., at least 3, at least 4, at least 5, at least 10, at least 15, at least 20, at least 25, at least 50, at least 75, at least 100 or more. In some embodiments, the term “a plurality of” refers to at least 100 or more, including, e.g., at least 250, at least 500, at least 750, at least 1000, or more. In some embodiments, the term “a plurality of” refers to at least 1000 or more, including, e.g., at least 1500, at least 2000, at least 3000, at least 4000, at least 5000, at least 7500, at least 10,000 or more.

The term “induced pluripotent stem cell” or “iPSC” or “iPS cell” refers to a cell derived from a complete reversion or reprogramming of the differentiation state of a differentiated cell (e.g. a somatic cell). As used herein, an iPSC is fully reprogrammed and is a cell which has undergone complete epigenetic reprogramming. As used herein, an iPSC is a cell which cannot be further reprogrammed (e.g., an iPSC cell is terminally reprogrammed).

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body-apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for reprogramming a differentiated cell can be performed both in vivo and in vitro (where in vivo is practiced when a differentiated cell is present within a subject, and where in vitro is practiced using isolated differentiated cell maintained in culture). In some embodiments, where a differentiated cell or population of differentiated cells are cultured in vitro, the differentiated cell can be cultured in an organotypic slice culture, such as described in, e.g., meneghel-Rozzo et al., (2004), Cell Tissue Res, 316(3); 295-303, which is incorporated herein in its entirety by reference.

All patents, patent applications, and publications identified herein are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1. Using One Embodiment of the Methods and/or Systems Described Herein to Predict Individualized Drug Response at the Gene Expression Level

In one aspect, presented herein relates to a computational algorithm for predicting individualized drug response at the gene expression level. In some embodiments, the inputs to the computational algorithm include, for example, but are not limited to an individual's pre-treatment gene expression profile (GEP) and known effects of the agent(s) (e.g., drug(s)) on gene expression in cells. The known effects of the agent(s) (e.g., drug(s)) on gene expressions in cells can be obtained through in vitro and/or cell-based assays and/or from publicly available in vitro and/or in vivo data. In some embodiments, the known effects (e.g., in vitro assay measurements and/or publicly available data) of the agent(s) (e.g., drug(s)) on gene expression in cells can be normalized, for example, to improve inter-assay and/or cross-data series comparability. Various art-recognized normalization methods can be used to normalize the known effects of the drug(s) on gene expression in cells. These examples rank normalization, quantile, LOESS, MASS, and RMA. In one embodiment, the normalization method can comprise rank normalization. For each sample, the genes are ranked according relative abundance, which is an indication of the degree to which each gene is expression. The rank, instead of the raw expression value, becomes the unit of analysis for further operations.

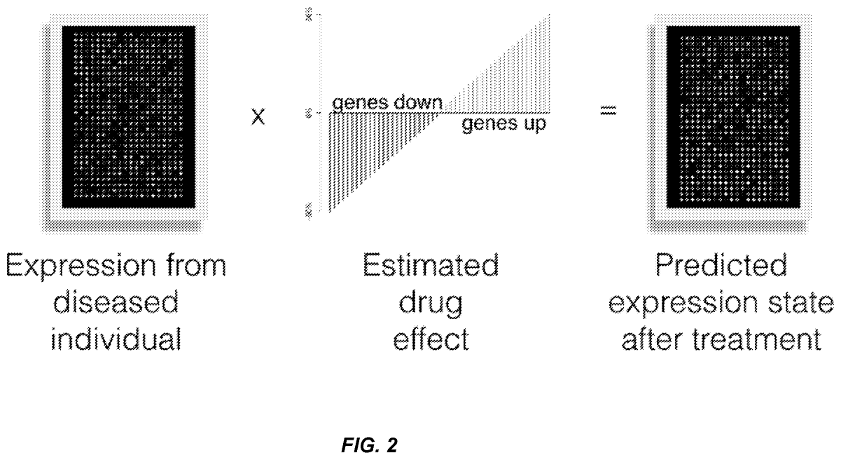

A new GEP for the individual, modulated by the input agent(s), can then be determined using the computation algorithm as a function of the aforementioned input parameters (FIG. 2). Accordingly, the computational algorithm and methods and/or systems described herein comprising the same can be used to predict how a specific person will respond to one or a plurality of (e.g., at least two) specific agents or drugs at granularity that is currently unavailable. FIG. 2 shows how the GEP from a diseased individual can be changed when the estimated effect of a specific drug is applied to the GEP. An exemplary system and/or method according to an embodiment of the present disclosure can then provide a predicted expression state after treatment. FIG. 2 shows how the original expression from the diseased individual and the predicted expression state after treatment can be examined to analyze the differences between the two expressions.

One of the major principles underlying the methods, systems, and/or computer readable media described herein is that a subject's response to one or more agents is best considered from a global, genome-wide perspective, instead of the traditional dichotomous approaches, where one or several genes are individually compared to a corresponding reference level without considering the interactions between the genes. Healthy people are typically characterized by a portion or subset of “gene-expression space” that is distinct from their non-healthy counterparts. Thus, if one could identify an agent or treatment that is determined to “nudge” a patient with a condition (e.g., a disease or disorder) towards the region of gene-expression space occupied by healthy patients, afflicted individuals are likely to experience some therapeutic benefit as a result. Accordingly, one aspect describes herein is a method for computationally estimating the direction and magnitude of this agent-induced “nudge” in an individual.

In one embodiment, the computational algorithm can compute a new expression level for each transcript in a patient's GEP if the patient were to be treated with one or more agent(s), as a function of the patient's pre-treatment GEP and known effects of the agent(s) (e.g., drug(s)) on gene expression in cells. By way of example only, assume g is a gene expression vector or matrix reflecting pre-treatment expression levels of at least 50% or more of the entire genome of an individual. In some embodiments, g can be a normalized expression value for some transcripts. The genes or transcripts selected for the analysis can include ones that are associated with a condition of the individual and/or targets of the agent(s). The expected post-treatment expression value for the transcripts, g_{new}, can be computed using the following equation:

g_{new}=g+g·dⱼ

wherein dⱼ is a transformation matrix reflecting known modulation of gene expression in cells associated with one or more agents of interest. In some embodiment, dⱼ can be a transformation matrix reflecting rank-normalized effect of the agent(s) on the transcripts g.

To demonstrate the viability of the approach, the computational algorithm was applied to patients with inflammatory bowel disease (IBD) using drugs that are routinely used in treatment of IBD. The pre-treatment GEP data on patients with IBD was obtained from an internal study of inflammatory bowel disease performed at Boston Children's hospital. The study collected colon biopsies from pediatric patients suspected of having IBD. Colon biopsies and whole blood samples were obtained. IBD status was determined from pathology reports on the colon biopsies, forming two cohorts of healthy controls (patients for whom the pathology report was negative) and confirmed cases of IBD (positive pathology reports). RNA was isolated from both diseased and healthy colon samples and measure using an Affymetrix ST 1.0 Gene array. The known effects of the agents on the IBD drugs on gene expressions were obtained through a public domain such as a gene expression data depository, e.g., a CMap project from the Broad Institute. The CMAP data was downloaded, normalized (see normalization section) and processed to remove batch and cell line effects. Additionally, the data were rank normalized.

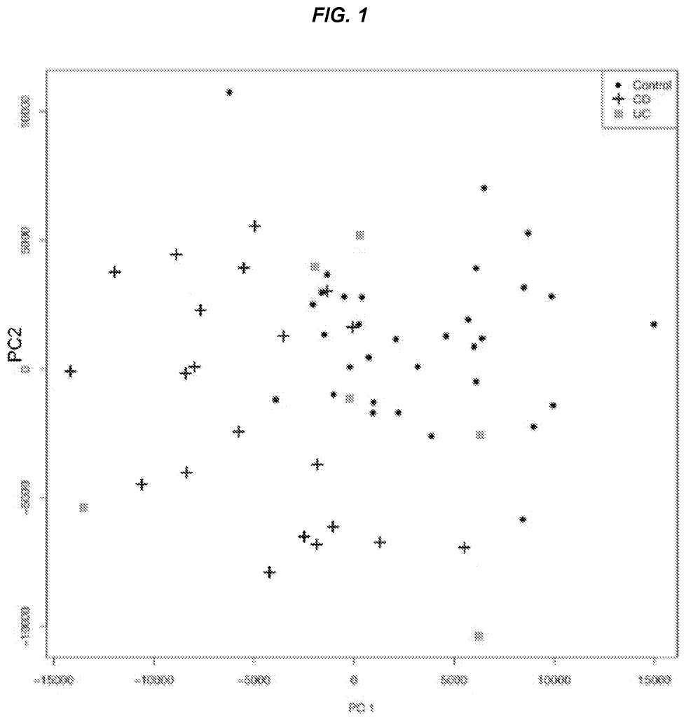

In some embodiments, principal component analysis (PCA) can be used to represent the gene expression profiles (GEP) or g. FIG. 1 shows a PCA plot of the GEP or g of patients with IBD and healthy controls. The healthy controls are healthy patients, individuals with Crohn's Disease are represented by CD, and individuals with Ulcerative Colitis are represented by UC. FIG. 1 shows clear separation of patients with IBD and controls (healthy patients), indicating that these patient populations can be well stratified in gene expression space.

Next, the dⱼ term reflecting known modulation of gene expression in cells associated with a IBD treatment, e.g., an immunosuppressant drug Azathioprine, was computationally applied to g to IBD patients available in the database using the computational algorithm described herein. Thus, the expected post-treatment gene expression profiles of the IBD patients or g_{new} can be determined. FIG. 3 shows a PCA plot of the predicted change in GEP induced by Azathioprine if the IBD patients were to be administered with Azathioprine. As shown in FIG. 3, the patients with ulcerative colitis (UC) and Crohn's disease (CD) move toward the gene expression space of healthy controls. The length of the arrow indicates the expected improvement for each patient, i.e. longer arrows indicate a patient will receive a more pronounced effect from the drug. Likewise, drugs may be given to healthy controls as well to estimate side-effects.

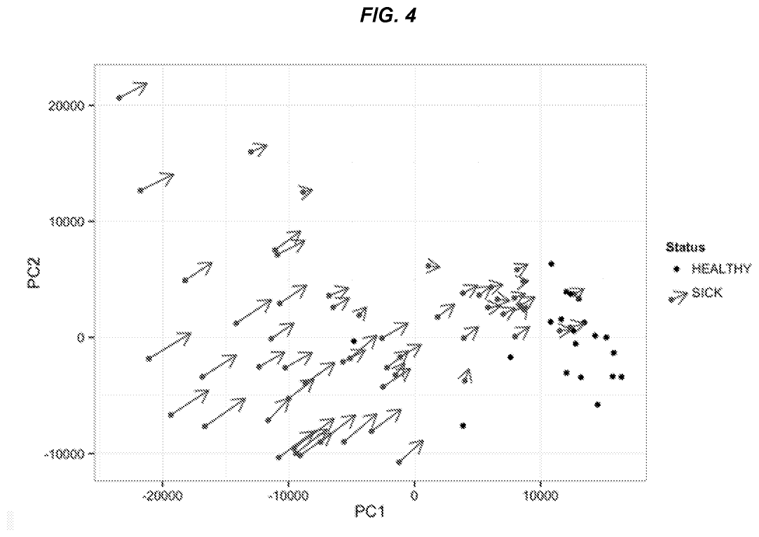

In addition to Azathioprine, the computational algorithm described herein was also validated in the same IBD dataset using several other well-known IBD treatments, e.g., sulfasalzine (FIG. 4) and negative controls (e.g., non-IBD treatments). FIG. 4 shows a PCA plot of predicted changes in GEP for IBD patients when treated with sulfasalazine. The majority of IBD patients, would benefit from sulfasalazine, as seen by how the arrows in the plot are largely directed towards the gene expression space of healthy controls. The length of the arrow indicates the expected improvement for each patient, i.e. longer arrows indicate a patient will receive a more pronounced effect from the drug.

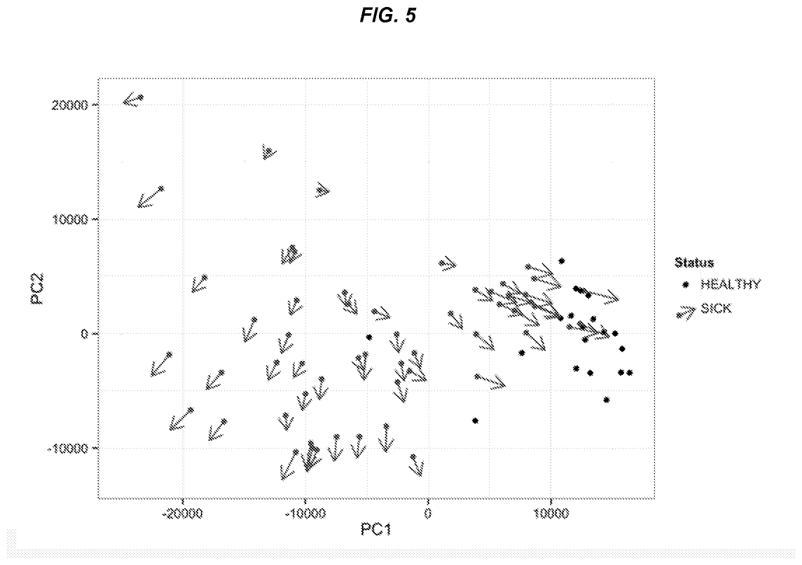

FIG. 5 shows a PCA plot of the predicted changes in GEP induced by another IBD treatment, e.g., prednisone, for IBD patients, indicating that in contrast to the PCA plot of an appropriate IBD treatment (e.g., in FIG. 4), a subset of the IBD patients are more likely to benefit from prednisone than others because only the subset of the IBD patients move toward the gene expression space of the healthy controls while the majority of the IBD patients appear to move away instead. As with FIGS. 3-4, in FIG. 5, the length of the arrow indicates the expected improvement for each patient, i.e. longer arrows indicate a patient will receive a more pronounced effect from the drug.

Additionally, the computational algorithm described herein was also validated in a separate, publicly available dataset in which GEPs of colon biopsies of IBD patients before and after treatment were available. The predicted GEP computed using the computational algorithm described herein agreed well with the GEPs of colon biopsies of the corresponding IBD patients after treatment, indicating that the algorithms described herein can robustly predict drug response. Resampling techniques (such as the bootstrap) can be used to estimate the variability associated with each prediction.

### Example 2. Using One Embodiment of the Methods and/or Systems Described Herein to Predict Drug Response at the Gene Expression Level on a Patient Database

A method, according to an exemplary embodiment of the present disclosure, can be used to identify a successful treatment method for a patient. Patient #1 was evaluated according to an exemplary methodology of the present disclosure. The methodology provided for collecting set of samples from Patient #1, including two inflamed colon samples, two non-inflamed colon samples, one inflamed bowel sample, one non-inflamed bowel sample, two inflamed esophagus samples, and two blood samples. These samples were all measured and replicated on 2 Affymetrix microarray platforms. Raw CEL files were obtain from the Helomics and processed using Affy Power Tools (APT). The patient samples along with raw reference data were simultaneously preprocessed and normalized using robust multi-array averaging (RMA) and quantile normalization. Probe level data was summarized to gene level by averaging probe values that mapped to the same Entrez gene ID.

After preliminary analysis, it was determined that the colon samples suffered from poor quality due to mRNA degradation resulting from being preserved in paraffin embedding for over 2 years. This made direct comparison to the reference dataset difficult. After exploratory analysis, it was decided that only genes that were differentially expressed in the reference dataset would be included in further analyses. Differential expression between healthy and IBD patients was computed using a gene-by-gene t-test and statistical significance was declared at a Bonferroni corrected level of 0.05.

All of the drug score analysis used only the patient's inflamed colon samples as the comparison set. The score for each drug was computed using the cosine similarity between two vectors representing the optimal treatment vector and the predicted treatment vector. The optimal treatment vector represents the “shortest distance” from the patient's current gene expression coordinates and the healthy patient centroid in the reference database.

A healthy centroid, C, can be defined as the average gene-wise expression profile for the healthy patients' colon samples. The patient's coordinates, P, can be defined as the gene-wise average of the patient's two inflamed colon samples. The optimal treatment vector, O, is then just the vector pointing from P to C, thus O=P−C.

Next, for a given drug, the methodology can estimate where this drug will push the patient on the basis of the drug's gene expression profile. This new point can be defined as P*, using the linear interpolation algorithm described previously. Finally, the drug's vector can be defined as D=P−P*. Interdrug potency can be obfuscated and prevent direct comparison of how “close” each drug pushes the patient to the healthy controls. However, an exemplary methodology according to the present disclosure can compare the how similar the optimal vector and each drug's predicted vector are by measuring the angle between them.

To compare the similarity between the optimal vector O, and a drug's vector D, we compute the cosine similarity defined as:

$$sim ( O , D ) = \frac{〈 O , D 〉}{\vert O \vert \vert D \vert}$$

<O,D> is an inner product and |O|,|D| are the vector norms. This measures the cosine of the angle between O and D and ranges from 1 if there is perfect agreement −1 if D is pointing in the opposite direction as O. A perfect agreement of 1 indicates that the selected drug will push the patient directly towards the healthy controls.

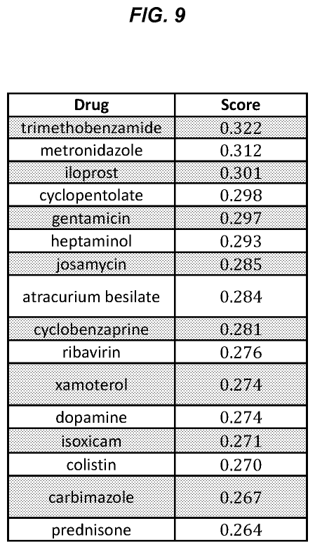

FIG. 9 shows a table generated according to an embodiment of the present disclosure where various drugs are scored to compare the optimal vector O and the drug's vector D. As mentioned previously, a score closer to 1 indicates that the drug will be a better fit for the patient. FIG. 9 shows that trimethobenzamide is predicted to have the best score.

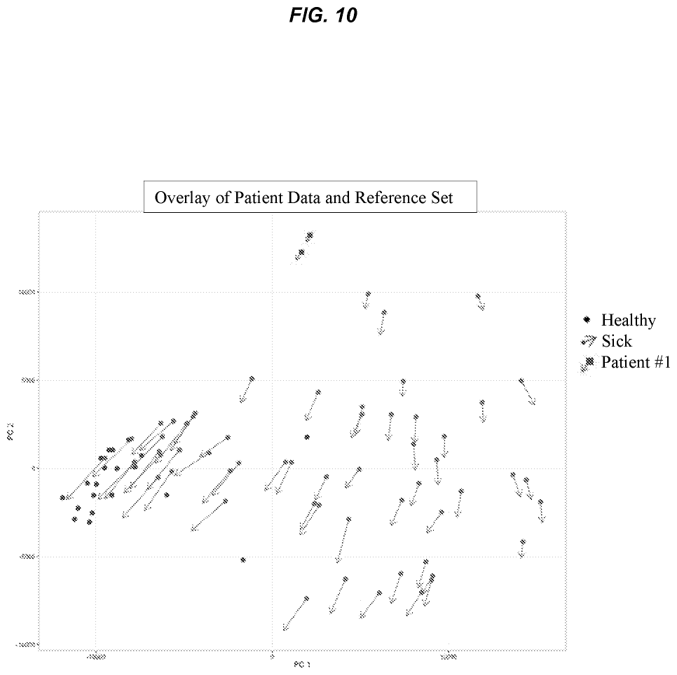

FIG. 10 is a PCA of predicted changes in GEP induced by trimethobenzamide in an exemplary patient. The predicted trajectory of the patient, shown by the crosses, moves towards the healthy patients (circles). Other exemplary patients with IBD are analyzed as well to show how they would be predicted to respond to the drug.

### Example 3. Using One Embodiment of the Methods and/or Systems Described Herein to Profile an Expected Patient's Response to Potential Drugs and Indicate a Preferred Treatment Plan

A method, according to an exemplary embodiment of the present disclosure, can be used to identify a successful treatment method for a patient where other attempted treatment methods have failed. For example, Patient #2 failed to receive effective treatment for pancolitis under conventional methods. Patient #2 was evaluated by a general intestinal doctor at three years old and ten months to receive treatment for bloody stool. At four years old, he underwent endoscopy which showed a pancolitis. Patient #2 was treated with sulfasalazine and fish oil until he was thirteen and a half years hold. He then started having 5-7 bowel movements per day some of which had bright red blood.

Several therapies under conventional methodologies were attempted without success. This included a glucocorticoid stress dose and a vancomycin treatment. The vancomycin treatment had a transient effect that lasted about a week. After 4 months he was started on 6-mercaptopurine with no improvement after 3 months. At this point Patient #2 became fatigued, partly because of the lack of sleep due to frequent bowel movements, and was no longer attending school and was being home tutored.

At approximately fourteen and a half years hold, he was started on infliximab 5 mg/kg with no effect. He was subsequently put on a course of Rifaximin, an antibiotic, which results in a transient improvement for a couple weeks. Two months later he was hospitalized as his bowel movements had become hourly and bloody. He was started on tacrolimus, an immunosuppressant, which caused a mild improvement but no remission. A colonoscopy of Patient #2 showed diffuse inflammation and this was confirmed on pathological examination of the mucosal biopsy. He was then started on Vedolizumab but Patient #2 did not show improvement even after more than six weeks of treatment with Vedolizumab. Eighteen months since the start of his flare, the pediatric general intestinal team was advocating for colectomy and the colorectal surgeon had scheduled surgery for the following month.

At that point, the tissue obtained in the most recent colonoscopic biospy was gene expression (RNA) profiled according to an exemplary methodology of the present disclosure. The methodology selected a drug which was predicted to maximally perturb the patient's transcriptome towards the centroid of the non-IBD group. In this case, the drug was indigo, an over the counter supplement, also known as Qing Dai. Patient #2 was started on indigo and within two weeks, his bowel movements had reduced in frequency to three to four per day and he was able to return to school. Fourteen months after regular treatment with indigo, Patient #2 has one to three bowel movements a day. Additionally, his growth in height, which had halted due to a combination of inflammation and drug therapy, has shown dramatic acceleration.

Therefore, this example shows the benefit of a treatment according to an exemplary embodiment of the present disclosure. Verifying a patient's gene expression profile before selecting a drug for treatment can correctly identify the best treatment plan for a patient without requiring the patient to undergo experimental periods of treatment with drugs that may not improve the patient's condition. As in this case, a team of pediatric doctors was unable to correctly identify an effective treatment for the patient, even after almost two years of attempted treatment. However, the present methodology selected an appropriate treatment plan for the patient on the first try.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present disclosure is not limited to the precise construction and compositions disclosed herein; any and all modifications, changes, and variations apparent from the foregoing descriptions are within the spirit and scope of the disclosure as defined in the appended claims. Moreover, the present concepts expressly include any and all combinations and sub combinations of the preceding elements and aspects. An implementation of an apparatus that falls within the inventive concept does not necessarily achieve any of the possible benefits outlined above: such benefits are dependent on the specific use case and specific implementation, and the possible benefits mentioned above are simply examples.

Although the concepts have been described above with respect to the various embodiments, it is noted that there can be a variety of permutations and modifications of the described features by those who are familiar with this field, only some of which have been presented above, without departing from the technical ideas and scope of the features, which is defined by the appended claims.

Further, while this specification contains many features, the features should not be construed as limitations on the scope of the disclosure or the appended claims. Certain features described in the context of separate embodiments can also be implemented in combination. Conversely, various features described in the context of a single embodiment can also be implemented in multiple embodiments separately or in any suitable sub-combination.

Although the drawings describe operations in a specific order and/or show specific arrangements of components, one should not interpret that such specific order and/or arrangements are limited, or that all the operations performed and the components disclosed are needed to obtain a desired result. There are numerous hardware and software devices that can be configured to forward data units in the manner described in the present disclosure with respect to various embodiments. Accordingly, other implementations are within the scope of the following claims.

What is claimed is:

1. A method, performed by a device comprising at least one processor, of selecting a treatment for a subject with a disease or disorder, the method comprising:
assaying, using at least one of said at least one processor, a sample from a subject with a disease or disorder to determine a pre-treatment genome-wide expression profile of the subject;
in a specifically-programmed computer, computing, for each of a library of gene-expression-modifying agents, an expected post-treatment genome-wide expression profile of the subject as a function of the pre-treatment genome-wide expression profile of the subject and known effects of the corresponding gene expression-modifying agent on gene expression in cells; and
identifying, using at least one of said at least one processor, a gene expression-modifying agent as an agent that is more likely to produce a therapeutic effect on the subject when deviation of the expected post-treatment genome-wide expression profile from a normal genome-wide expression profile is smaller than deviation of the pre-treatment genome-wide expression profile from the normal genome-wide expression profile; or
identifying, using at least one of said at least one processor, a gene expression-modifying agent as an agent that is less likely to produce a therapeutic effect on the subject when deviation of the expected post-treatment genome-wide expression profile from a normal genome-wide expression profile is greater than or substantially same as deviation of the pre-treatment genome-wide expression profile from the normal genome-wide expression profile, thereby selecting a treatment comprising a gene expression-modifying agent that is personalized to the subject.

2. The method of claim 1, wherein the computing comprises principal component analysis (PCA) of the pre-treatment genome-wide expression profile and the normal genome-wide expression profile to identify a set of gene signatures that are associated with the disease or disorder of the subject.

3. The method of claim 2, wherein the library of gene expression-modifying agents are selected for the computing based on their known properties to modulate expression of at least one of the gene signatures toward its corresponding expression level in normal cells not affected by the disease or disorder.

4. The method of claim 2 or 3, wherein the expected post-treatment genome-wide expression profile of the subject is computed using the following equation:
g_{new}=g+g·dⱼ
wherein g is a gene expression vector reflecting at least a subset of genes of the pre-treatment genome-wide expression profile of the subject, wherein the subset of genes correspond to the gene signatures associated with the disease or disorder; dⱼ is a transformation matrix reflecting a known modulation of gene expression in cells associated with each of the gene expression-modifying agents; and g_{new} is a gene expression matrix reflecting an expected post-treatment genome-wide expression profile for each of the gene expression-modifying agents.

5. The method of any of claims 1-4, wherein the sample is assayed by a method comprising polymerase chain reaction (PCR), a real-time quantitative PCR, microarray, and nucleic acid sequencing.

6. The method of any of claims 1-5, further comprising administering to the subject a gene expression-modifying agent identified to be more likely to produce a therapeutic effect on the subject.

7. The method of claim 6, wherein the administered gene expression-modifying agent has not been clinically known for treatment of the disease or disorder.

8. The method of any of claims 1-5, further comprising administering to the subject an alternative treatment when the gene expression-modifying agent is identified to be less likely to produce a therapeutic effect on the subject.

9. The method of any of claims 1-8, wherein the disease or disorder is an inflammatory bowel disease.

10. A method, performed by a device comprising at least one processor, of treating a subject with a disease or disorder, the method comprising:
administering to a subject with a disease or disorder a treatment that is computationally selected to be more likely to modulate the genome-wide expression profile of the subject toward a normal genome-wide expression profile, wherein the computational drug selection process comprises:
computing, using at least one of said at least one processor, for each of a library of gene-expression-modifying agents, an expected post-treatment genome-wide expression profile of the subject as a function of a pre-treatment genome-wide expression profile of the subject and known effects of the corresponding gene expression-modifying agent on gene expression in cells; and
identifying, using at least one of said at least one processor, a gene expression-modifying agent as an agent that is more likely to produce a therapeutic effect on the subject when deviation of the expected post-treatment genome-wide expression profile from a normal genome-wide expression profile is smaller than deviation of the pre-treatment genome-wide expression profile from the normal genome-wide expression profile; or
identifying, using at least one of said at least one processor, a gene expression-modifying agent as an agent that is less likely to produce a therapeutic effect on the subject when deviation of the expected post-treatment genome-wide expression profile from a normal genome-wide expression profile is greater than or substantially same as deviation of the pre-treatment genome-wide expression profile from the normal genome-wide expression profile.

11. The method of claim 10, wherein the administered gene expression-modifying agent has not been clinically known for treatment of the disease or disorder.

12. The method of claim 10 or 11, wherein the treatment comprises at least one gene expression-modifying agent that is identified in the computational drug selection process to be more likely to produce a therapeutic effect.

13. The method of claim 10 or 11, wherein the treatment comprises a combination treatment comprising at least two gene expression-modifying agents, wherein the combination treatment is identified in the computational drug selection process to be more likely to produce a therapeutic effect.

14. A method, performed by a device comprising at least one processor, of identifying a subject who is diagnosed with a disease or disorder and is more likely to respond to a treatment, the method comprising:
assaying, using at least one of said at least one processor, a sample from the subject to determine a genome-wide expression profile of the subject;
computing, using at least one of said at least one processor, an expected post-treatment genome-wide expression profile of the subject as a function of the pre-treatment genome-wide expression profile of the subject and known effects of the treatment on gene expression in cells; and
identifying, using at least one of said at least one processor, the subject to be more likely to respond to the treatment when deviation of the expected post-treatment genome-wide expression profile from a normal genome-wide expression profile is smaller than deviation of the pre-treatment genome-wide expression profile from the normal genome-wide expression profile; or
identifying, using at least one of said at least one processor, the subject to be likely to respond to an alternative treatment when deviation of the expected post-treatment genome-wide expression profile from a normal genome-wide expression profile is greater than or substantially similar to deviation of the pre-treatment genome-wide expression profile from the normal genome-wide expression profile.

15. A method, performed by a device comprising at least one processor, of drug repositioning, the method comprising:
obtaining, using at least one of said at least one processor, individual genome-wide expression profiles of patients identified with the same disease or disorder;
for each identified patient, computing, using at least one of said at least one processor, an expected post-treatment genome-wide expression profile of the identified patient as a function of the corresponding individual genome-wide expression profile and known effects of a therapeutic agent on gene expression, wherein the therapeutic agent is not clinically known to be indicated for treatment of the disease or disorder identified in the patients; and
identifying, using at least one of said at least one processor, the therapeutic agent as an agent that is likely to produce a therapeutic effect on the disease or disorder identified in the patients when at least 50% or more of the patients show the expected post-treatment genome-wide expression profile with a smaller deviation from a normal genome-wide expression profile than that of the individual genome-wide expression profile from the normal genome-wide expression profile, thereby computationally repositioning the therapeutic agent for a new indication; or
identifying, using at least one of said at least one processor, the therapeutic as an agent that is not likely to produce a therapeutic effect on the disease or disorder identified in the patients when less than 50% of the patients show the expected post-treatment genome-wide expression profile with a smaller deviation from a normal genome-wide expression profile than that of the individual genome-wide expression profile from the normal genome-wide expression profile.

16. The method of claim 15, further comprising, when the therapeutic agent is computationally repositioned for a new indication, contacting cells in vitro or in an animal model with the therapeutic agent to experimentally validate its therapeutic effect, wherein the cells in vitro or in animal model correspond to a model of the same disease or disorder as identified in the patients.

17. A method, performed by a device comprising at least one processor, of identifying a potential adverse effect of a treatment in a subject with a disease or disorder, the method comprising:
assaying, using at least one of said at least one processor, a sample from the subject to determine a pre-treatment genome-wide expression profile of the subject;
computing, using at least one of said at least one processor, an expected post-treatment genome-wide expression profile of the subject as a function of the pre-treatment genome-wide expression profile of the subject and known effects of the treatment on gene expression in cells; and
identifying, using at least one of said at least one processor, the treatment to be more likely to induce an adverse effect in the subject when deviation of the expected post-treatment genome-wide expression profile from a normal genome-wide expression profile is larger than deviation of the pre-treatment genome-wide expression profile from the normal genome-wide expression profile, and/or the expected post-treatment genome-wide expression profile of the subject is similar to expected post-treatment genome-wide expression profiles of patients who have suffered from at least one adverse effect upon administration of the same treatment; or
identifying, using at least one of said at least one processor, the treatment to be less likely to induce an adverse effect when deviation of the expected post-treatment genome-wide expression profile from a normal genome-wide expression profile is smaller than deviation of the pre-treatment genome-wide expression profile from the normal genome-wide expression profile, and/or the expected post-treatment genome-wide expression profile of the subject is different from expected post-treatment genome-wide expression profiles of patients who have suffered from at least one adverse effect upon administration of the same treatment.
